(19) United States
(12) Patent Application Publication
(10) Pub. No.: US 2023/0346772 A1
(43) Pub. Date: Nov. 2, 2023

(54) COMBINATION TREATMENTS FOR WALDENSTROM'S MACROGLOBULINEMIA

(71) Applicant: X4 Pharmaceuticals, Inc., Boston, MA (US)

(72) Inventor: E. Lynne KELLEY, Cambridge, MA (US)

(21) Appl. No.: 17/786,912

(22) PCT Filed: Dec. 18, 2020
(86) PCT No.: PCT/US20/66099

§ 371 Date: Jun. 17, 2022

Related U.S. Application Data
(60) Provisional application No. 62/949,968, filed on Dec. 18, 2019.

(51) Int. Cl. A61K 31/4709; A61K 31/519

(52) U.S. Cl. CPC A61K 31/4709; A61K 31/519

(57) ABSTRACT

The present invention relates to methods of treating cancer, in which a CXCR4 inhibitor such as X4P-001 or a pharmaceutically acceptable salt thereof or pharmaceutical composition thereof is administered in combination with an additional therapeutic agent, such as a BTK inhibitor. Accordingly, in one aspect, the present invention provides a method of treating Waldenstrom's macroglobulinemia, comprising administering to a patient in need thereof an effective amount of a CXCR4 inhibitor such as X4P-001, or a pharmaceutically acceptable salt thereof, and co-administering simultaneously or sequentially an effective amount of one or more additional therapeutic agents, such as those described herein.

### FIELD OF THE INVENTION

The present invention relates to methods for treating cancer, such as combination therapies comprising a CXCR4 inhibitor and a BTK inhibitor. The cancer includes lymphomas such as Waldenstrom's macroglobulinemia.

### CROSS-REFERENCE TO RELATED APPLICATIONS

This application claims the benefit of U.S. Provisional Application No. 62/949,968, filed on Dec. 18, 2019, the entirety of which is hereby incorporated by reference.

### BACKGROUND OF THE INVENTION

Waldenstrom's macroglobulinemia (WM) is a distinct B-cell lymphoproliferative disorder characterized by the proliferation of lymphoplasmacytic cells in the bone marrow in other organs, along with elevated serum levels of monoclonal immunoglobulin M (IgM) gammopathy (Owen 2003; Treon 2013). Waldenstrom's macroglobulinemia represents a spectrum from early asymptomatic monoclonal gammopathy of undetermined significance (MGUS) (IgM-MGUS), where there are small numbers of lymphoplasmacytic cells in the bone marrow (<10%), to active WM with anemia, hyperviscosity, and widespread disease (Kyle 2004; Kyle 2005). In a similar plasma-cell dyscrasia such as multiple myeloma, studies have demonstrated the presence of a small number of circulating plasma cells in over 70% of patients with multiple myeloma (Nowakowski 2005). The number of circulating cells in the peripheral blood increased with progression of the disease and was an independent unfavorable prognostic marker (Nowakowski 2005). This data implies that progression of these plasma cell dyscrasias occurs through the continuous trafficking of the malignant cells to new sites of the bone marrow (Nowakowski 2005).

The World Health Organization classification defines WM as lymphoplasmacytic lymphoma with overexpression of a clone of IgM proteins, belonging to the category of non-Hodgkin B cell lymphomas with a typically indolent course.

Waldenstrom's macroglobulinemia accounts for approximately 2% of all cases of non-Hodgkin lymphoma. It presents with distinctive clinical and laboratory features related to the presence of the monoclonal IgM (Mazzucchelli 2018).

Clinical manifestation of WM includes symptoms associated with anemia (e.g., pallor, weakness, fatigue), systemic complaints (e.g., weight loss, fever, night sweats), organomegaly (e.g., enlarged lymph nodes, spleen, and/or liver), and/or symptoms related to the IgM monoclonal protein in the blood (e.g., hyperviscosity, peripheral neuropathy, and cryoglobulinemia) requiring various degrees of urgency in diagnosis and treatment to avoid secondary complications (Dimopoulos 2000).

These data underscore the significant, unmet need for study of therapies, including combination therapies, to treat rare cancers such as WM. The present invention addresses this need and provides certain other related advantages.

### SUMMARY OF THE INVENTION

It has now been found that CXCR4 inhibitors such as X4P-001, in combination with a BTK inhibitor such as ibrutinib, are useful in treating a variety of cellular proliferative disorders, such as those described herein.

CXCR4 inhibitors such as the compound X4P-001, or a pharmaceutically acceptable salt thereof, as described in greater detail below, are useful as a combination therapy with one or more additional therapeutic agents described herein, such as a BTK inhibitor. Accordingly, in one aspect, the present invention provides a method of treating Waldenstrom's macroglobulinemia, comprising administering to a patient in need thereof an effective amount of a CXCR4 inhibitor such as X4P-001, or a pharmaceutically acceptable salt thereof, and co-administering simultaneously or sequentially an effective amount of one or more additional therapeutic agents, such as those described herein.

### DETAILED DESCRIPTION OF CERTAIN EMBODIMENTS OF THE INVENTION

Waldenstrom's macroglobulinemia (WM) is a distinct B-cell lymphoproliferative disorder characterized by the proliferation of lymphoplasmacytic cells in the bone marrow in other organs, along with elevated serum levels of monoclonal immunoglobulin M (IgM) gammopathy (Owen 2003; Treon 2013).

WM is sometimes referred to as a lymphoplasmacytic lymphoma (LPL) with an associated monoclonal IgM paraprotein. In WM, there is a malignant change to the B-cell in the late stages of maturing, and it continues to proliferate into a clone of identical cells, primarily in the bone marrow but also in the lymph nodes and other tissues and organs of the lymphatic system.

Under the microscope, WM cells have characteristics of both B-lymphocytes and plasma cells, and they are called lymphoplasmacytic cells. For that reason, WM is classified as a type of non-Hodgkin's lymphoma called lymphoplasmacytic lymphoma (LPL). About 95% of LPL cases are WM; the remaining 5% do not secrete IgM and consequently are not classified as WM. WM is a very rare disease—only about 1,500 patients are diagnosed with it each year in the US. For reference, approximate normal levels of IgM are described, e.g., in Gonzalez-Quíntela et al. (2007) Clinical and Experimental Immunology 151: 42-50. Normal levels are approximately: 70 mg/190 ml for males; and 80-250 mg/100 ml for females. See also “Range of normal serum immunoglobulin (IgG, IgA and IgM) values in Nigerians,” Oyeyinko et al., Afr J Med Med Sci 1984, September-December; 13(3-4):169-76. Mean values of IgM varied from 65 to 132 mg/100 ml in the males and from 96 to 114 mg/100 ml in the females.

For men, normal hemoglobin levels are about 13.5 to 17.5 grams per deciliter; for women, 12.0 to 15.5 grams per deciliter.

As a result of proliferation in the bone marrow and other sites, the lymphoplasmacytic cells of WM may interfere with normal functioning. In the bone marrow where blood cells are produced, the WM cells “crowd out” the normal blood cells and may lead to a reduction in normal blood counts; in the lymph nodes and other organs, the WM cells may lead to enlargement of these structures and other complications.

Somatic mutation in myeloid differentiation primary response 88 (MYD88) is found in over 90% of patients with WM. Mutations in chemokine (C-X-C motif) receptor 4 (CXCR4) are the next most common mutations and found to be present in 43% of patients with WM (Xu 2016). Published pivotal clinical studies conducted with ibrutinib have reported MYD88 and CXCR4 mutation status affected responses to ibrutinib. In WM, three genomic groups have been delineated on the basis of clinical manifestations and survival: 1) MYD88^{L265} CXCR4^{WT} [with WT indicating wild type], 2) MYD88^{L265P}CXCR4^{WHIM} [with WHIM indicating warts, hypogammaglobulinemia, infections, and myelokathexis], and 3) MYD88^{WT}CXCR4^{WT}. WHIM-like mutations result in a gain of function in CXCR4, which in turn decreases chemokine (C-X-C motif) 12 (CXCL12) mediated receptor down regulation and ultimately inhibits egress of cells bearing the mutant CXCR4 from sequestered areas in bone marrow and lymph nodes (Lei 2016; Majumdar 2018). These findings affirm an important role for MYD88 and CXCR4 somatic mutations in the pathogenesis of tumors (Treon 2015 [2]). In the ibrutinib published studies in treatment-naïve and previously treated WM patients, the very good partial response (VGPR) and major response (defined as complete response+VGPR+partial response) rates were highest among patients with MYD88^{L265P}CXCR4^{WT} and significantly lower for those with MYD88^{L265P}CXCR4^{WHIM}. The major response rates and VGPR rates are summarized in the Table below. The CXCR4^{WHIM} mutations have been associated with more aggressive disease features, such as higher IgM levels and bone marrow involvement.

TABLE 1
Ibrutinib Outcome by Mutational Status
in Waldenstrom's Macroglobulinemia

| | | All | MYD88^{L265P} | MYD88^{L265P} |
|---|---|---|---|---|
| | Response | Patients | CXCR4^{WT} | CXCR4^{WHIM} |
| Ibrutinib Monotherapy | MR¹ | 78% | 97% | 67% |
| in Previously Treated | VGPR¹ | 29% | 44% | 10% |
| WM Patients | | | | |
| Ibrutinib (Open-Label) | MR² | 83% | 94% | 71% |
| in Treatment- Naïve | VGPR² | 20% | 31% | 7% |
| WM Patients | | | | |

Abbreviations:
CXCR4 = chemokine (C-X-C motif) receptor 4;
MR = major response;
MYD88 = myeloid differentiation primary response 88;
WHIM = warts, hypogammaglobulinemia, infections, and myelokathexis;
VGPR = very good partial response;
vs = versus;
WM = Waldenstrom's macroglobulinemia;
WT = wild type.
¹Source = Treon 2015.
²Source = Treon 2018.

Other somatic mutations associated with WM, although with substantially lower frequencies of about 3%-10%, have been identified. These include CD79B; HIST1HH1E; MYBPPlA, ARID1A; HISTIHHIB; TP53; and MLL2, as well as patients with more than one mutation, including TP53/CD79B; RAG2/ARID1A; IST1IHHI1E/IST1IHHI1B. Jiminez et al. (2015) Blood 126:2971; Poulain et al. (2016) Blood 128:4092; Hunter et al. (2014) Blood 123:1637; each of which is hereby incorporated by reference.

In some embodiments, a disclosed method comprises treatment of a patient having WM that bears a somatic mutation in the CXCR4 receptor. Without wishing to be bound by theory, it is believed that because patients with this mutation have a lesser response to ibrutinib, co-administration with a CXCR4 inhibitor such as mavorixa for will provide improved treatment outcomes for such patients.

Chemokines are major regulators of cell trafficking and adhesion. The chemokine CXCL12 (stromal cell-derived factor-1α) is normally expressed on hematopoietic cells such as hematopoietic stem cells (HSCs), T cells, B cells, monocytes and macrophages, neutrophils, and eosinophils (Chatterjee 2014; Nagase 2000). CXCL12 has potent chemotactic activity for lymphocytes and myeloid-derived suppressor cells and is important in homing of HSCs to the bone marrow. When CXCL12 activates CXCR4, it enhances and sustains AKT, extracellular signal-regulated kinase, and Bruton's tyrosine kinase (BTK) signaling pathways, as well as increases cell migration, adhesion, growth, and survival of WM cells (Cao 2014). The chemokine CXCL12 binds primarily to CXC receptor 4 (CXCR4; CD184). The binding of CXCL12 to CXCR4 induces intracellular signaling through several divergent pathways initiating signals related to chemotaxis, cell survival and/or proliferation, increase in intracellular calcium, and gene transcription. CXCR4 is expressed on multiple cell types including lymphocytes, HSCs, endothelial and epithelial cells, and cancer cells. The CXCL12/CXCR4 axis is involved in tumor progression, angiogenesis, metastasis, and survival. This pathway is a target for the development of therapeutic agents that can block the CXCL12/CXCR4 interaction or inhibit downstream intracellular signaling.

In WHIM syndrome, the gain-of-function mutation in CXCR4 results in decreased release of leukocytes into the bloodstream. Treatment with a CXCR4 antagonist has been shown to mobilize leukocytes to beneficially impact the characteristic lymphopenia and leukopenia observed in WHIM patients (Liu 2015; Dale 2011).

At least 40 different nonsense and frameshift mutations in the C-terminal domain of CXCR4 have been described in WM (Poulain 2016; Xu 2016; Stone 2004) The nonsense mutations truncate the distal 15- to 20 amino acid region and the frameshift mutations comprise a region of up to 40 amino acids in the C-terminal domain (Hunter 2014). Nonsense and frameshift mutations are almost equally divided among WM patients. The most common CXCR4 mutation in WM is a nonsense mutation of S338X. The presence of CXCR4 somatic mutations can affect disease presentation in WM. Patients with CXCR4 mutations present with a significantly lower rate of adenopathy, and those with CXCR4 nonsense mutations have an increased bone marrow disease burden, serum IgM levels, and/or risk of symptomatic hyperviscosity (Stone 2004; Treon 2014).

Without wishing to be bound by theory, it is believed that, given the important role of the CXCR4^{WHIM} mutation in ibrutinib-resistance in both clinical and preclinical studies, as well as the other anti-tumor activities of a CXCR4 inhibitor, the effective CXCR4 antagonism by X4P-001 (mavorixafor) provides a significant benefit in patients with WM.

The incidence of WM is approximately 3 per million people per year with 1400 new cases diagnosed in the United States (US) each year (Fonseca 2007; Groves 1998). The median age at the time of diagnosis is 70 years. Less than 10% of patients are under 50 years of age, and approximately 60% are males (Castillo 2015; Pophali 2018). Waldenstrom's macroglobulinemia is much more common in Caucasians than in other ethnic groups. Specifically, it is uncommon in blacks, who make up approximately 5% of cases, and those of Mexican descent (Fonseca 2007).

In a study published by Varettoni, the median treatment-free survival was significantly shorter in asymptomatic WM patients harboring a CXCR4 mutation at diagnosis (median 51 months) than in those with wild type CXCR4 (median not reached). In multivariate analysis, CXCR4 mutation was an independent prognostic factor for progression from asymptomatic to symptomatic WM requiring therapy (Varettoni 2017).

Waldenstrom's macroglobulinemia patients are often treated with rituximab, an anti-CD20 antibody, as monotherapy or in combination with alkylating agents (bendamustine and cyclophosphamide) or nucleoside analogues (fludarabine and cladribine). Other novel therapies include BTK inhibitors (such as ibrutinib, acalabrutinib and zanubrutinib), proteasome inhibitors (bortezomib and carfilzomib), thalidomide, and everolimus (Buske 2013; Dimopoulos 2014; Treon 2015 [2]; Owen 2014; Dimopoulos 2007; Olszewski 2016). Even though these treatments show some activity, they are not curative, and a standard of care has not been established for WM (Dimopoulos 2017). Therefore, new and/or additional therapeutic options are needed.

Ibrutinib has been approved as a single agent to treat WM in both US and European Union (EU) (ibrutinib (IMBRUVICA®)). In the US, ibrutinib can be used in any line of treatment while in the EU, ibrutinib is approved for patients who have received at least one prior therapy, or in first-line treatment for patients unsuitable for chemo-immunotherapy. The ibrutinib monotherapy and rituximab combination pivotal trials have identified genetic mutation patients who have not benefited to the same extent of those patients without genetic mutations. One specific population identified with a remaining unmet medical need is the double mutation, MYD88^{L265P} CXCR4^{WHIM} population, estimated at approximately 27% of the WM population (Treon 2015 [1], Treon 2018 [1]; Hunter 2014). Patients with the double mutations had a significantly reduced VGPR (9.5%) as compared with patients with the MYD88^{L265P} CXCR4^{WT} mutations (44.4%) (Treon 2015 [2]; Treon 2018 [2]).

X4P-001 (mavorixafor) is an orally bioavailable, small molecule inhibitor of CXCR4. It has now been found that CXCR4 inhibitors such as X4P-001, or a pharmaceutically acceptable salt thereof or pharmaceutical composition thereof, as described in greater detail below, is useful as a combination therapy with one or more other therapeutic agents described herein. Accordingly, in one aspect, the present invention provides a method of treating a cancer, such as those described herein, by administering to a patient in need thereof an effective amount of X4P-001, or a pharmaceutically acceptable salt thereof or pharmaceutical composition thereof, and comprising co-administering simultaneously or sequentially an effective amount of one or more additional therapeutic agents, such as those described herein. In some embodiments, the method includes co-administering one additional therapeutic agent. In some embodiments, the method includes co-administering two additional therapeutic agents. In some embodiments, the combination of X4P-001 and the additional therapeutic agent or agents acts synergistically to prevent or reduce immune escape and/or angiogenic escape of the cancer. In some embodiments, the patient has previously been administered another anticancer agent, such as an adjuvant therapy or immunotherapy. In some embodiments, the cancer is refractory.

In some embodiments, X4P-001 or a pharmaceutically acceptable salt thereof or pharmaceutical composition thereof is used in combination with an approved cancer therapy such as radiation, a chemotherapeutic, or an immunotherapy or targeted therapeutic. In some embodiments, the approved cancer therapy is chemotherapy, a targeted drug, a biological therapy, plasmapheresis (plasma exchange), stem cell transplant, or radiation therapy.

In one aspect, the present invention provides a method of treating Waldenstrom's macroglobulinemia (WM) in a patient in need thereof, comprising administering to the patient an effective amount of X4P-001, or a pharmaceutically acceptable salt thereof, in combination with an effective amount of a BTK inhibitor, or a pharmaceutically acceptable salt thereof, and wherein the WM is selected from one of the following genomic groups: 1) MYD88^{L265} CXCR4^{WT} [with WT indicating wild type], 2) MYD88^{L265P}CXCR4^{WHIM} [with WHIM indicating warts, hypogammaglobulinemia, infections, and myelokathexis], and 3) MYD88^{WT}CXCR4^{WT}. In some embodiments, the WM comprises cells of two, or all three, genomic groups.

Additional agents that may be co-administered with X4P-001 are described in WO 2018/237158, the entire contents of which are hereby incorporated by reference.

In some embodiments, the co-administered agent is a BTK inhibitor. In some embodiments, the BTK inhibitor is ibrutinib, acalabrutinib, or zanubrutinib.

The chemical name for ibrutinib is 1-[(3R)-3-[4-amino-3-(4-phenoxyphenyl)-1Hpyrazolo[3,4-d]pyrimidin-1-yl]-1-piperidinyl]-2-propen-1-one and has the following structure:

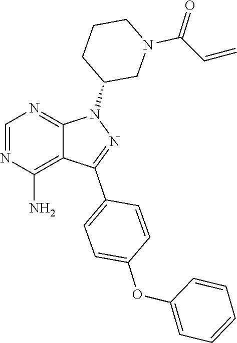 [SMILES: C=CC(=O)N1CCC[C@@H](n2nc(-c3ccc(Oc4ccccc4)cc3)c3c(N)ncnc32)C1]

In the United States, IMBRUVICA® (ibrutinib) capsules for oral administration are available in the following dosage strengths: 70 mg and 140 mg. Each capsule contains ibrutinib (active ingredient) and the following inactive ingredients: croscarmellose sodium, magnesium stearate, microcrystalline cellulose, sodium lauryl sulfate. The capsule shell contains gelatin, titanium dioxide, yellow iron oxide (70 mg capsule only), and black ink. Ibrutinib tablets for oral administration are available in the following dosage strengths: 140 mg, 280 mg, 420 mg, and 560 mg. Each tablet contains ibrutinib (active ingredient) and the following inactive ingredients: colloidal silicon dioxide, croscarmellose sodium, lactose monohydrate, magnesium stearate, microcrystalline cellulose, povidone, and sodium lauryl sulfate. The film coating for each tablet contains ferrosoferric oxide (140 mg, 280 mg, and 420 mg tablets), polyvinyl alcohol, polyethylene glycol, red iron oxide (280 mg and 560 mg tablets), talc, titanium dioxide, and yellow iron oxide (140 mg, 420 mg, and 560 mg tablets).

Ibrutinib (Ibruvica® Pharmacyclics; AbbVie) is approved for:
Treatment of mantle cell lymphoma in adult patients who have received at least one prior therapy.
Treatment of chronic lymphocytic leukemia (CLL) or small lymphocytic lymphoma (SLL) [both including CLL and SLL with 17p deletion
Treatment of Waldenström's macroglobulinemia.
Marginal zone lymphoma (MZL) who require systemic therapy and have received at least one prior anti-CD20-based therapy
Chronic graft versus host disease (cGVHD) after failure of one or more lines of systemic therapy

Dosage:
MCL and MZL: 560 mg taken orally once daily.
CLL/SLL, WM, and cGVHD: 420 mg taken orally once daily.

Dose should be taken orally with a glass of water. Do not open, break, or chew the capsules. Do not cut, crush, or chew the tablets.

In the United States, acalabrutinib (Calquence® AstraZeneca Pharmaceuticals) is approved for:
Treatment of mantle cell lymphoma in adult patients who have received at least one prior therapy;
Treatment of chronic lymphocytic leukemia (CLL) or small lymphocytic lymphoma (SLL).

Recommended dose is 100 mg orally approximately every 12 hours; swallow whole with water and with or without food.

In the United States, zanubrutinib (Brukinsa® Beigene, USA) is approved for:
Treatment of mantle cell lymphoma in adult patients who have received at least one prior therapy.

Recommended dose: 160 mg orally twice daily or 320 mg orally once daily; swallow whole with water and with or without food. Reduce dose in patients with severe hepatic impairment.

In one aspect, the present invention provides a method of treating Waldenstrom's macroglobulinemia (WM) in a patient in need thereof, comprising administering to the patient an effective amount of X4P-001, or a pharmaceutically acceptable salt thereof, in combination with an effective amount of a BTK inhibitor, or a pharmaceutically acceptable salt thereof, and wherein the WM bears one or more somatic mutations in the CXCR4 gene.

In another aspect, the present invention provides a method of determining whether a patient's WM will respond to treatment, comprising:
(a) testing a biological sample taken from the patient for a CXCR4 mutation and optionally a MYD88 mutation;
(b) if the patient's WM bears at least one CXCR4 mutation, selecting the patient for treatment with a CXCR4 inhibitor.

In some embodiments, the method further comprises the step of treating the patient with a combination of an effective amount of X4P-001, or a pharmaceutically acceptable salt thereof; and an effective amount of a BTK inhibitor, or a pharmaceutically acceptable salt thereof.

In another aspect, the present invention provides a method of treating Waldenstrom's macroglobulinemia (WM) bearing MYD88^{L265P}CXCR4^{WHIM} and/or MYD88^{WT}CXCR^{WHIM} mutations in a patient in need thereof, comprising administering to the patient an effective amount of X4P-001, or a pharmaceutically acceptable salt thereof; in combination with an effective amount of a BTK inhibitor, or a pharmaceutically acceptable salt thereof.

In some embodiments, the one or more somatic mutations comprise a gain of function mutation relative to wild type CXCR4.

In some embodiments, the one or more somatic mutations comprise a WHIM-like mutation that results in a gain of function in CXCR4 relative to wild type CXCR4.

In some embodiments, the one or more somatic mutations comprise a CXCR4(S338X) somatic mutation.

In some embodiments, the WM further comprises a somatic MYD88 mutation and optionally a somatic deletion associated with B-cell lymphomagenesis.

In some embodiments, the MYD88 mutation is MYD88^{L265P}.

In some embodiments, the BTK inhibitor is ibrutinib, acalabrutinib, or zanubrutinib.

In some embodiments, the BTK inhibitor is ibrutinib.

In some embodiments, the patient has previously received at least one course of treatment with a BTK inhibitor, or a pharmaceutically acceptable salt thereof, before treatment with X4P-001, or a pharmaceutically acceptable salt thereof.

In some embodiments, the patient is treatment naïve, i.e., the patient has not received a previous treatment for WM. In some embodiments, the patient has not received previous treatment with a BTK inhibitor (such as ibrutinib), or a pharmaceutically acceptable salt thereof. In some embodiments, the patient has not received previous treatment with a BTK inhibitor (such as ibrutinib), or a pharmaceutically acceptable salt thereof, and has not received previous treatment with X4P-001, or a pharmaceutically acceptable salt thereof.

In some embodiments, the patient's WM is resistant to treatment with a BTK inhibitor.

In some embodiments, the patient has previously received at least one course of treatment with a BTK inhibitor, such as ibrutinib, acalabrutinib, or zanubrutinib, before treatment with X4P-001 or a pharmaceutically acceptable salt thereof.

In some embodiments, the patient's WM has shown disease progression.

In some embodiments, X4P-001 or a pharmaceutically acceptable salt thereof is administered to the patient in a dose of about 100 mg to about 1000 mg per day.

In some embodiments, X4P-001 or a pharmaceutically acceptable salt thereof is administered to the patient in a dose of about 200 mg to about 600 mg per day.

In some embodiments, X4P-001 or a pharmaceutically acceptable salt thereof is administered to the patient in a dose of about 200 mg, about 400 mg, or about 600 mg per day.

In some embodiments, X4P-001 or a pharmaceutically acceptable salt thereof is administered to the patient in a single daily dose (QD).

In some embodiments, X4P-001 or a pharmaceutically acceptable salt thereof and ibrutinib or a pharmaceutically acceptable salt thereof are administered to the patient in a fasted state.

In some embodiments, ibrutinib or a pharmaceutically acceptable salt thereof is administered to the patient in a dose of about 70 mg to about 840 per day.

In some embodiments, ibrutinib or a pharmaceutically acceptable salt thereof is administered to the patient in a dose of about 140 mg to about 420 mg per day.

In some embodiments, ibrutinib or a pharmaceutically acceptable salt thereof is administered to the patient in a dose of about 140 mg, about 280 mg, or about 420 mg per day.

In some embodiments, X4P-001 is administered to the patient in a dose of about 200 mg to about 600 mg per day in combination with ibrutinib in a dose of about 140 mg to about 420 mg per day.

In some embodiments, X4P-001 is administered to the patient in a dose of about 200 mg, about 400 mg, or about 600 mg per day in combination with ibrutinib in a dose of about 140 mg, about 280 mg, or about 420 mg per day.

In some embodiments, X4P-001 is administered to the patient in a dose of about 200 mg per day in combination with ibrutinib in a dose of about 140 mg, about 280 mg, or about 420 mg per day.

In some embodiments, X4P-001 is administered to the patient in a dose of about 400 mg per day in combination with ibrutinib in a dose of about 140 mg, about 280 mg, or about 420 mg per day.

In some embodiments, X4P-001 is administered to the patient in a dose of about 600 mg per day in combination with ibrutinib in a dose of about 140 mg, about 280 mg, or about 420 mg per day.

In some embodiments, X4P-001 is administered to the patient in a dose of about 200 mg per day in combination with ibrutinib in a dose of about 140 mg per day.

In some embodiments, X4P-001 is administered to the patient in a dose of about 400 mg per day in combination with ibrutinib in a dose of about 280 mg per day.

In some embodiments, X4P-001 is administered to the patient in a dose of about 600 mg per day in combination with ibrutinib in a dose of about 420 mg per day.

In some embodiments, the method provides at least a 50% percent decrease in IgM levels from baseline. In some embodiments, the method provides at least a 10%, 20%, 25%, 30%, 35%, 40%, 45%, 50%, 60%, 70%, 75%, 80%, 85%, 90%, 95%, or 98% decrease in IgM levels from baseline. In some embodiments, the method provides about a 50% decrease in IgM levels from baseline. In some embodiments, the method provides about a 10%, 20%, 25%, 30%, 35%, 40%, 45%, 50%, 60%, 70%, or 75% decrease in IgM levels from baseline. In some embodiments, the method provides about a 10-20%, 10-25%, 15-30%, 15-35%, 20-40%, 20-45%, 25-50%, 30-60%, 35-70%, 50-60%, 50-75%, 60-90%, 70-90%, 80-90%, 80-95%, 80-98%, 85-98%, 90-98%, or 95-98% decrease in IgM levels from baseline.

In some embodiments, the method reduces IgM and/or Hgb to within 2 times the normal range for a non-diseased adult human (non-WM patient), 1.5 times, 1.25 times, or to within the normal range for a non-diseased adult human.

In some embodiments, the method decreases Hgb to between 2 times the upper limit of normal (ULN) and the lower limit of normal.

In some embodiments, X4P-001, or a pharmaceutically acceptable salt thereof, and the BTK inhibitor, or a pharmaceutically acceptable salt thereof, act synergistically.

In some embodiments, the dose of the BTK inhibitor, or a pharmaceutically acceptable salt thereof, required for effective treatment is decreased by at least 20% relative to the effective dose of the BTK inhibitor, or a pharmaceutically acceptable salt thereof, as a monotherapy.

In some embodiments, the method further comprises administering an additional therapeutic agent, such as rituximab or another described herein.

In some embodiments, the method further comprises the step of obtaining a biological sample from the patient and measuring the amount of a disease-related biomarker.

In some embodiments, the biological sample is a blood sample.

In some embodiments, the disease-related biomarker is selected from circulating CD8+ T cells or the ratio of CD8+ T cells:Treg cells.

In some embodiments, the disease-related biomarker is IgM and/or Hgb. In some embodiments, the biomarker is absolute neutrophil count (ANC).

In some embodiments, the additional therapeutic agent is an immunostimulatory therapeutic compound.

In some embodiments, the immunostimulatory therapeutic compound is selected from elotuzumab, mifamurtide, an agonist or activator of a toll-like receptor, or an activator of RORγt.

In some embodiments, the method further comprises administering to said patient an additional therapeutic agent, such as an immune checkpoint inhibitor. In some embodiments, the method comprises administering to the patient in need thereof three therapeutic agents selected from X4P-001 or a pharmaceutically acceptable salt thereof, a BTK inhibitor, and an immunostimulatory therapeutic compound or immune checkpoint inhibitor.

In some embodiments, the immune checkpoint inhibitor is selected from nivolumab, pembrolizumab, ipilimumab, avelumab, durvalumab, atezolizumab, or pidilizumab.

In some embodiments, the additional therapeutic agents are selected from an indoleamine (2,3)-dioxygenase (IDO) inhibitor, a Poly ADP ribose polymerase (PARP) inhibitor, a histone deacetylase (HDAC) inhibitor, a CDK4/CDK6 inhibitor, or a phosphatidylinositol 3 kinase (PI3K) inhibitor.

In some embodiments, the IDO inhibitor is selected from epacadostat, indoximod, capmanitib, GDC-0919, PF-06840003, BMS.F001287, Phy906/KD108, or an enzyme that breaks down kynurenine.

In some embodiments, the PARP inhibitor is selected from olaparib, rucaparib, or niraparib.

In some embodiments, the HDAC inhibitor is selected from vorinostat, romidepsin, panobinostat, belinostat, entinostat, or chidamide.

In some embodiments, the CDK 4/6 inhibitor is selected from palbociclib, ribociclib, abemaciclib or trilaciclib.

In some embodiments, the method further comprises administering to said patient a third therapeutic agent, such as an immune checkpoint inhibitor. In some embodiments, the method comprises administering to the patient in need thereof three therapeutic agents selected from X4P-001 or a pharmaceutically acceptable salt thereof, a BTK inhibitor, and a third therapeutic agent selected from an indoleamine (2,3)-dioxygenase (IDO) inhibitor, a Poly ADP ribose polymerase (PARP) inhibitor, a histone deacetylase (HDAC) inhibitor, a CDK4/CDK6 inhibitor, or a phosphatidylinositol 3 kinase (PI3K) inhibitor, and an immune checkpoint inhibitor. In some embodiments, the immune checkpoint inhibitor is selected from nivolumab, pembrolizumab, ipilimumab, avelumab, durvalumab, atezolizumab, or pidilizumab.

In some embodiments, the PI3K inhibitor is selected from idelalisib, alpelisib, taselisib, pictilisib, copanlisib, duvelisib, PQR309, or TGR1202.

In some embodiments, the method further comprises administering to said patient a platinum-based therapeutic, a taxane, a nucleoside inhibitor, or a therapeutic agent that interferes with normal DNA synthesis, protein synthesis, cell replication, or will otherwise inhibit rapidly proliferating cells.

In some embodiments, the platinum-based therapeutic is selected from cisplatin, carboplatin, oxaliplatin, nedaplatin, picoplatin, or satraplatin.

In some embodiments, the taxane is selected from paclitaxel, docetaxel, albumin-bound paclitaxel, cabazitaxel, or SID530.

In some embodiments, the therapeutic agent that interferes with normal DNA synthesis, protein synthesis, cell replication, or will otherwise interfere with the replication of rapidly proliferating cells is selected from trabectedin, mechlorethamine, vincristine, temozolomide, cytarabine, lomustine, azacitidine, omacetaxine mepesuccinate, asparaginase Erwinia chrysanthemi, eribulin mesylate, capacetrine, bendamustine, ixabepilone, nelarabine, clorafabine, trifluridine, or tipiracil.

In some embodiments, the patient has a solid tumor. Solid tumors generally comprise an abnormal mass of tissue that typically does not include cysts or liquid areas. In some embodiments, the cancer is Waldenström's macroglobulinemia.

In some embodiments, the patient has a resectable solid tumor, meaning that the patient's tumor is deemed susceptible to being removed by surgery. In other embodiments, the patient has an unresectable solid tumor, meaning that the patient's tumor has been deemed not susceptible to being removed by surgery, in whole or in part.

In some embodiments, the present invention provides a method for treating refractory cancer in a patient in need thereof comprising administering to a patient in need thereof an effective amount of X4P-001 or a pharmaceutically acceptable salt thereof or pharmaceutical composition thereof, in combination with a BTK inhibitor and optionally further in combination with an additional therapeutic agent such as those described herein.

In certain embodiments, the patient was previously administered a protein kinase inhibitor. In some embodiments, the patient was previously administered a VEGF-R antagonist. In certain embodiments, the patient was previously administered an immune checkpoint inhibitor. In some embodiments, the patient was previously administered an immune checkpoint inhibitor selected from nivolumab (Opdivo®, Bristol-Myers Squibb), pembrolizumab (Keytruda®, Merck), or ipilumumab (Yervoy®, Bristol-Myers Squibb).

In some embodiments, X4P-001, or a pharmaceutically acceptable salt thereof or pharmaceutical composition thereof, is administered to a patient in a fasted state.

### Co-Administered Therapeutic Agents

In certain embodiments, X4P-001 or a pharmaceutically acceptable salt thereof, or another CXCR4 antagonist, is administered in combination with an additional therapeutic agent. In some embodiments, X4P-001 or a pharmaceutically acceptable salt thereof, or another CXCR4 antagonist, is administered in combination with one additional therapeutic agent. In some embodiments, X4P-001 or a pharmaceutically acceptable salt thereof, or another CXCR4 antagonist, is administered in combination with two additional therapeutic agents. In some embodiments, X4P-001 or a pharmaceutically acceptable salt thereof, or another CXCR4 antagonist, is administered in combination with three or more additional therapeutic agents.

In some embodiments, the additional therapeutic agent is a kinase inhibitor or VEGF-R antagonist. Approved VEGF inhibitors and kinase inhibitors useful in the present invention include: bevacizumab (Avastin®, Genentech/Roche) an anti-VEGF monoclonal antibody; ramucirumab (Cyramza®, Eli Lilly), an anti-VEGFR-2 antibody and ziv-aflibercept, also known as VEGF Trap (Zaltrap®; Regeneron/Sanofi). VEGFR inhibitors, such as regorafenib (Stivarga®, Bayer); vandetanib (Caprelsa®, AstraZeneca); axitinib (Inlyta®, Pfizer); and lenvatinib (Lenvima®, Eisai); Raf inhibitors, such as sorafenib (Nexavar®, Bayer AG and Onyx); dabrafenib (Tafinlar®, Novartis); and vemurafenib (Zelboraf®, Genentech/Roche); MEK inhibitors, such as cobimetanib (Cotellic®, Exelexis/Genentech/Roche); trametinib (Mekinist®, Novartis); Bcr-Abl tyrosine kinase inhibitors, such as imatinib (Gleevec®, Novartis); nilotinib (Tasigna®, Novartis); dasatinib (Sprycel®, BristolMyersSquibb); bosutinib (Bosulif®, Pfizer); and ponatinib (Inclusig®, Ariad Pharmaceuticals); Her2 and EGFR inhibitors, such as gefitinib (Iressa®, AstraZeneca); erlotinib (Tarceeva®, Genentech/Roche/Astellas); lapatinib (Tykerb®, Novartis); afatinib (Gilotrif®, Boehringer Ingelheim); osimertinib (targeting activated EGFR, Tagrisso®, AstraZeneca); and brigatinib (Alunbrig®, Ariad Pharmaceuticals); c-Met and VEGFR2 inhibitors, such as cabozanitib (Cometriq®, Exelexis); and multikinase inhibitors, such as sunitinib (Sutent®, Pfizer); pazopanib (Votrient®, Novartis); ALK inhibitors, such as crizotinib (Xalkori®, Pfizer); ceritinib (Zykadia®, Novartis); and alectinib (Alecenza®, Genentech/Roche); Bruton's tyrosine kinase inhibitors, such as ibrutinib (Imbruvica®, Pharmacyclics/Janssen); and Flt3 receptor inhibitors, such as midostaurin (Rydapt®, Novartis).

Other kinase inhibitors and VEGF-R antagonists that are in development and may be used in the present invention include tivozanib (Aveo Pharmaecuticals); vatalanib (Bayer/Novartis); lucitanib (Clovis Oncology); dovitinib (TKI258, Novartis); Chiauanib (Chipscreen Biosciences); CEP-11981 (Cephalon); linifanib (Abbott Laboratories); neratinib (HKI-272, Puma Biotechnology); radotinib (Supect®, IY5511, Il-Yang Pharmaceuticals, S. Korea); ruxolitinib (Jakafi®, Incyte Corporation); PTC299 (PTC Therapeutics); CP-547,632 (Pfizer); foretinib (Exelexis, GlaxoSmithKline); quizartinib (Daiichi Sankyo) and motesanib (Amgen/Takeda).

### Exemplary Standard of Care Therapies

### Waldenström's Macroglobulinemia

In some embodiments, the present invention provides a method of treating Waldenström's macroglobulinemia in a patient in need thereof, comprising administering to the patient an effective amount of X4P-001 in combination with an effective amount of a BTK inhibitor, and optionally further in combination with one or more standard of care treatments, or a combination thereof, for Waldenström's macroglobulinemia.

Standard of care treatments for Waldenström's macroglobulinemia are well known to one of ordinary skill in the art and include chemotherapy, or immunotherapy, or a combination thereof. In some embodiments, the standard of care chemotherapy is selected from chlorambucil, cladribine, cyclophosphamide, fludarabine, bendamustine, or a BTK inhibitor, such as ibrutinib, acalabrutinib, or zanubrutinib. In some embodiments, the additional therapeutic agent is ibrutinib (Imbruvica®; Pharmacyclics/Janssen/AbbVie).

In some embodiments, X4P-001 is administered to the patient as a monotherapy and as the first-line treatment for the Waldenström's macroglobulinemia. In other embodiments, X4P-001 is administered to the patient as a first-line treatment in combination with a standard of care treatment for Waldenström's macroglobulinemia (e.g., immunotherapy, or chemotherapy, or a combination thereof).

In some embodiments, when a standard of care treatment fails, such as when the Waldenström's macroglobulinemia is partially resistant to a chemotherapy, a second-line treatment is used that can include a well-known second-line treatment to treat Waldenström's macroglobulinemia. Accordingly, in some embodiments, the present invention provides a method of treating Waldenström's macroglobulinemia in a patient wherein the cancer is resistant to a first-line therapy, said method comprising administering X4P-001 optionally in combination with a second-line treatment.

In some embodiments, the present invention provides a method of treating a resistant Waldenström's macroglobulinemia comprising administering X4P-001 as the second-line treatment. In some embodiments, the present invention provides a method of treating a resistant Waldenström's macroglobulinemia comprising administering X4P-001 in combination with another second-line treatment or standard of care second-line treatment for Waldenström's macroglobulinemia (e.g., immunotherapy, chemotherapy, etc.). In some embodiments, the second-line treatment is selected from a chemotherapy. For example, X4P-001 is administered as a second-line therapy in combination with a chemotherapy for the treatment of relapsed and refractory Waldenström's macroglobulinemia.

In some instances when the first-line or second-line standard of care treatment fails, such as when chemotherapy continues to fail and remission occurs, a third-line treatment is administered to the patient that can include a well-known third-line treatment to treat Waldenström's macroglobulinemia. In some embodiments, the present invention provides a method of treating a Waldenström's macroglobulinemia resistant to both first-line therapy and second-line therapy comprising administering X4P-001 as the third-line treatment. In some embodiments, the present invention provides a method of treating a Waldenström's macroglobulinemia resistant to both first-line therapy and second-line therapy comprising administering X4P-001 in combination with another third-line treatment or standard of care third-line treatment for Waldenstrom's macroglobulinemia (e.g., immunotherapy, chemotherapy, etc.).

In some embodiments, X4P-001 is administered as a sensitizer for the treatment of Waldenström's macroglobulinemia. Without wishing to be bound by any particular theory, it is believed that X4P-001 increases the efficacy of the standard of care, first-line, second-line, or third-line treatments for Waldenström's macroglobulinemia, wherein the Waldenström's macroglobulinemia comprises a CXCR4 mutation such as one of those described herein. In some embodiments, the present invention provides a method of treating a Waldenström's macroglobulinemia in a patient in need thereof, comprising administering X4P-001 to the patient prior to administration of one or more of a standard of care, first-line, second-line, or third-line treatment. In some embodiments, administration of X4P-001 results in a more effective treatment of the Waldenström's macroglobulinemia compared to treatment of Waldenström's macroglobulinemia in the absence of administration of X4P-001. In some embodiments, the present invention provides a method of treating a Waldenström's macroglobulinemia in a patient in need thereof, comprising administering X4P-001 to the patient after administration of one or more of a standard of care, first-line, second-line, or third-line treatment.

In some embodiments, the present invention provides a method of treating a Waldenström's macroglobulinemia in a patient in need thereof, comprising administering X4P-001 to the patient in combination with an additional therapeutic agent suitable for treating the Waldenström's macroglobulinemia. In some embodiments, the additional therapeutic agent is a BTK inhibitor. In some embodiments, the additional therapeutic agent is selected from chlorambucil, cladribine, cyclophosphamide, fludarabine, bendamustine, and ibrutinib. In some embodiments, the additional therapeutic agent is ibrutinib (Imbruvica®; Pharmacyclics/Janssen/AbbVie).

One of ordinary skill in the art will understand the amount and dosing regimen to administer such additional therapeutic agents for the treatment of Waldenström's macroglobulinemia. By way of example, the administration of exemplary therapeutic agents suitable for treating Waldenström's macroglobulinemia is summarized in Table 2, below.

TABLE 2
Exemplary Therapies for Waldenström's Macroglobulinemia

| Therapeutic Agent | Dosing regimen |
|---|---|
| ibrutinib | 420 mg taken orally once daily for |
| (Imbruvica ®; | WM until disease progression or |
| Pharmacyclics/ | unacceptable toxicity. |
| Janssen/AbbVie) | Doses taken with a glass of water. |

In some embodiments, the present invention provides a method of treating a cancer in a patient in need thereof, as described herein, comprising administering to the patient X4P-001 in combination with one or more additional therapies wherein the combination of X4P-001 and the one or more additional therapies acts synergistically. In some embodiments, the administration of X4P-001 in combination with an additional therapeutic agent results in a reduction of the effective amount of that additional therapeutic agent as compared to the effective amount of the additional therapeutic agent in the absence of administration in combination with X4P-001. In some embodiments, the effective amount of the additional therapeutic agent administered in combination with X4P-001 is about 90%, about 80%, about 70%, about 60%, about 50%, about 40%, about 30%, about 20%, or about 10% of the effective amount of the additional therapeutic agent in the absence of administration in combination with X4P-001.

As used herein, “about” means that the stated value or range may vary by up to 10% from the stated value or range. For example, “about” 5.0 means 5.0±0.5, and “about 5.0-10.0” means 4.5-10.5.

### Dosage and Formulations

X4P-001 is a CXCR4 antagonist, with molecular formula C₂₁H₂₇N₅; molecular weight 349.48 amu; and appearance as a white to pale yellow solid. Solubility: freely soluble in the pH range 3.0 to 8.0 (>100 mg/mL), sparingly soluble at pH 9.0 (10.7 mg/mL) and slightly soluble at pH 10.0 (2.0 mg/mL). X4P-001 is only slightly soluble in water. Melting point: 108.9° C.

The chemical structure of X4P-001 is depicted below.

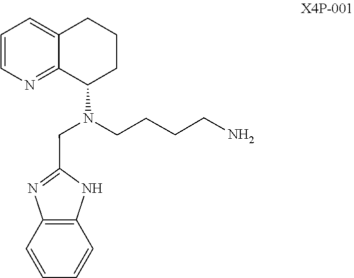 [SMILES: NCCCCN(Cc1nc2ccccc2[nH]1)[C@H]1CCCc2cccnc21]

In certain embodiments, a pharmaceutical composition containing X4P-001 or a pharmaceutically acceptable salt thereof is administered orally in an amount from about 200 mg to about 1200 mg daily. In certain embodiments, the dosage composition may be provided twice a day in divided dosage, approximately 12 hours apart. In other embodiments, the dosage composition may be provided once daily. The terminal half-life of X4P-001 has been generally determined to be between about 12 to about 24 hours, or approximately 14.5 hrs. Dosage for oral administration may be from about 100 mg to about 1200 mg once or twice per day. In certain embodiments, the dosage of X4P-001 or a pharmaceutically acceptable salt thereof useful in the invention is from about 200 mg to about 600 mg daily. In other embodiments, the dosage of X4P-001 or a pharmaceutically acceptable salt thereof useful in the invention may range from about 400 mg to about 800 mg, from about 600 mg to about 1000 mg or from about 800 mg to about 1200 mg daily. In certain embodiments, the invention comprises administration of an amount of X4P-001 or a pharmaceutically acceptable salt thereof of about 10 mg, about 20 mg, about 25 mg, about 50 mg, about 75 mg, about 100 mg, about 125 mg, about 150 mg, about 200 mg, about 250 mg, about 300 mg, about 400 mg, about 450 mg, about 500 mg, about 600 mg, about 650 mg, about 700 mg, about 750 mg, about 800 mg, about 850 mg, about 900 mg, about 950 mg, about 1000 mg, about 1100 mg, about 1200 mg, about 1300 mg, about 1400 mg, about 1500 mg, or about 1600 mg.

In some embodiments, a provided method comprises administering to the patient a pharmaceutically acceptable composition comprising X4P-001 or a pharmaceutically acceptable salt thereof wherein the composition is formulated for oral administration. In certain embodiments, the composition is formulated for oral administration in the form of a tablet or a capsule. In some embodiments, the composition comprising X4P-001 or a pharmaceutically acceptable salt thereof is formulated for oral administration in the form of a capsule.

In certain embodiments, a provided method comprises administering to the patient one or more unit doses, such as capsules, comprising 100-1200 mg X4P-001 or a pharmaceutically acceptable salt thereof as an active ingredient; and one or more pharmaceutically acceptable excipients.

A composition according to the present invention comprises a compound for use in the invention or a pharmaceutically acceptable salt or derivative thereof and a pharmaceutically acceptable carrier, adjuvant, or vehicle. The amount of compound in compositions of this invention is an amount effective to measurably inhibit CXCR4, or a mutant thereof, in a biological sample or in a patient. In certain embodiments, a composition of this invention is formulated for administration to a patient in need of such a composition. In some embodiments, a composition of this invention is formulated for oral administration to a patient.

The term “patient,” as used herein, means an animal, preferably a mammal, and most preferably a human.

As used herein, the term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, S. M. Berge et al., describe pharmaceutically acceptable salts in detail in J. Pharmaceutical Sciences, 1977, 66, 1-19, incorporated herein by reference. Pharmaceutically acceptable salts of the compounds of this invention include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, pivalate, propionate, stearate, succinate, sulfate, tartrate, thiocyanate, p-toluenesulfonate, undecanoate, valerate salts, and the like.

Salts derived from appropriate bases include alkali metal, alkaline earth metal, ammonium and N⁺(C₁₋₄alkyl)₄ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, loweralkyl sulfonate and aryl sulfonate.

The term “pharmaceutically acceptable carrier, adjuvant, or vehicle” refers to a non-toxic carrier, adjuvant, or vehicle that does not destroy the pharmacological activity of the compound with which it is formulated. Pharmaceutically acceptable carriers, adjuvants or vehicles that may be used in the compositions of this invention include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

A “pharmaceutically acceptable derivative” means any non-toxic salt, ester, salt of an ester or other derivative of a compound of this invention that, upon administration to a patient, is capable of providing, either directly or indirectly, a compound of this invention.

Compositions of the present invention may be administered orally, parenterally, by inhalation spray, topically (as by powders, ointments, or drops), rectally, nasally, buccally, intravaginally, intracisternally, or via an implanted reservoir. The term “parenteral” as used herein includes subcutaneous, intravenous, intramuscular, intra-articular, intra-synovial, intrasternal, intrathecal, intrahepatic, intralesional, and intracranial injection or infusion techniques. Preferably, the compositions are administered orally, intraperitoneally or intravenously. Sterile injectable forms of the compositions of this invention may be aqueous or oleaginous suspension. These suspensions may be formulated according to techniques known in the art using suitable dispersing or wetting agents and suspending agents. The sterile injectable preparation may also be a sterile injectable solution or suspension in a non-toxic parenterally acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium.

For this purpose, any bland fixed oil may be employed including synthetic mono- or di-glycerides. Fatty acids, such as oleic acid and its glyceride derivatives are useful in the preparation of injectables, as are natural pharmaceutically acceptable oils, such as olive oil or castor oil, especially in their polyoxyethylated versions. These oil solutions or suspensions may also contain a long-chain alcohol diluent or dispersant, such as carboxymethyl cellulose or similar dispersing agents that are commonly used in the formulation of pharmaceutically acceptable dosage forms including emulsions and suspensions. Other commonly used surfactants, such as Tweens, Spans and other emulsifying agents or bioavailability enhancers which are commonly used in the manufacture of pharmaceutically acceptable solid, liquid, or other dosage forms may also be used for the purposes of formulation.

Pharmaceutically acceptable compositions of this invention may be orally administered in any orally acceptable dosage form including, but not limited to, capsules, tablets, aqueous suspensions or solutions. In the case of tablets for oral use, carriers commonly used include lactose and corn starch. Lubricating agents, such as magnesium stearate, are also typically added. For oral administration in a capsule form, useful diluents include lactose and dried cornstarch. When aqueous suspensions are required for oral use, the active ingredient is combined with emulsifying and suspending agents. If desired, certain sweetening, flavoring or coloring agents may also be added.

Alternatively, pharmaceutically acceptable compositions of this invention may be administered in the form of suppositories for rectal administration. These can be prepared by mixing the agent with a suitable non-irritating excipient that is solid at room temperature but liquid at rectal temperature and therefore will melt in the rectum to release the drug. Such materials include cocoa butter, beeswax and polyethylene glycols.

Pharmaceutically acceptable compositions of this invention may also be administered topically, especially when the target of treatment includes areas or organs readily accessible by topical application, including diseases of the eye, the skin, or the lower intestinal tract. Suitable topical formulations are readily prepared for each of these areas or organs.

Topical application for the lower intestinal tract can be effected in a rectal suppository formulation (see above) or in a suitable enema formulation. Topically-transdermal patches may also be used.

For topical applications, provided pharmaceutically acceptable compositions may be formulated in a suitable ointment containing the active component suspended or dissolved in one or more carriers. Carriers for topical administration of compounds of this invention include, but are not limited to, mineral oil, liquid petrolatum, white petrolatum, propylene glycol, polyoxyethylene, polyoxypropylene compound, emulsifying wax and water. Alternatively, provided pharmaceutically acceptable compositions can be formulated in a suitable lotion or cream containing the active components suspended or dissolved in one or more pharmaceutically acceptable carriers. Suitable carriers include, but are not limited to, mineral oil, sorbitan monostearate, polysorbate 60, cetyl esters wax, cetearyl alcohol, 2-octyldodecanol, benzyl alcohol and water.

For ophthalmic use, provided pharmaceutically acceptable compositions may be formulated as micronized suspensions in isotonic, pH adjusted sterile saline, or, preferably, as solutions in isotonic, pH adjusted sterile saline, either with or without a preservative such as benzylalkonium chloride. Alternatively, for ophthalmic uses, the pharmaceutically acceptable compositions may be formulated in an ointment such as petrolatum.

Pharmaceutically acceptable compositions of this invention may also be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other conventional solubilizing or dispersing agents.

Most preferably, pharmaceutically acceptable compositions of this invention are formulated for oral administration. Such formulations may be administered with or without food. In some embodiments, pharmaceutically acceptable compositions of this invention are administered without food. In other embodiments, pharmaceutically acceptable compositions of this invention are administered with food.

The amount of compounds of the present invention that may be combined with the carrier materials to produce a composition in a single dosage form will vary depending upon the host treated and the particular mode of administration. Preferably, provided compositions should be formulated so that a dosage of between 0.01-100 mg/kg body weight/day of the inhibitor can be administered to a patient receiving these compositions.

It should also be understood that a specific dosage and treatment regimen for any particular patient will depend upon a variety of factors, including the activity of the specific compound employed, the age, body weight, general health, sex, diet, time of administration, rate of excretion, drug combination, and the judgment of the treating physician and the severity of the particular disease being treated. The amount of a compound of the present invention in the composition will also depend upon the particular compound in the composition.

The compounds and compositions, according to the method of the present invention, may be administered using any amount and any route of administration effective for treating a cancer, such as those disclosed herein. The exact amount required will vary from subject to subject, depending on the species, age, and general condition of the subject, the severity of the cancer, the particular agent, its mode of administration, and the like. Compounds of the invention are preferably formulated in dosage unit form for ease of administration and uniformity of dosage. The expression “dosage unit form” as used herein refers to a physically discrete unit of agent appropriate for the patient to be treated. It will be understood, however, that the total daily usage of the compounds and compositions of the present invention will be decided by the attending physician within the scope of sound medical judgment. The specific effective dose level for any particular patient or organism will depend upon a variety of factors including the cancer being treated and the severity of the cancer; the activity of the specific compound employed; the specific composition employed; the age, body weight, general health, sex and diet of the patient; the time of administration, route of administration, and rate of excretion of the specific compound employed; the duration of the treatment; drugs used in combination or coincidental with the specific compound employed, and like factors well known in the medical arts.

In certain embodiments, the compounds of the invention may be administered orally or parenterally at dosage levels of about 0.01 mg/kg to about 50 mg/kg and preferably from about 1 mg/kg to about 25 mg/kg, of subject body weight per day, one or more times a day, to obtain the desired therapeutic effect.

Liquid dosage forms for oral administration include, but are not limited to, pharmaceutically acceptable emulsions, microemulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Injectable preparations, for example, sterile injectable aqueous or oleaginous suspensions may be formulated according to the known art using suitable dispersing or wetting agents and suspending agents. The sterile injectable preparation may also be a sterile injectable solution, suspension or emulsion in a nontoxic parenterally acceptable diluent or solvent, for example, as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid are used in the preparation of injectables.

Injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

In order to prolong the effect of a compound of the present invention, it is often desirable to slow the absorption of the compound from subcutaneous or intramuscular injection. This may be accomplished by the use of a liquid suspension of crystalline or amorphous material with poor water solubility. The rate of absorption of the compound then depends upon its rate of dissolution that, in turn, may depend upon crystal size and crystalline form. Alternatively, delayed absorption of a parenterally administered compound form is accomplished by dissolving or suspending the compound in an oil vehicle. Injectable depot forms are made by forming microencapsule matrices of the compound in biodegradable polymers such as polylactide-polyglycolide. Depending upon the ratio of compound to polymer and the nature of the particular polymer employed, the rate of compound release can be controlled. Examples of other biodegradable polymers include poly(orthoesters) and poly(anhydrides). Depot injectable formulations are also prepared by entrapping the compound in liposomes or microemulsions that are compatible with body tissues.

Compositions for rectal or vaginal administration are preferably suppositories which can be prepared by mixing the compounds of this invention with suitable non-irritating excipients or carriers such as cocoa butter, polyethylene glycol or a suppository wax which are solid at ambient temperature but liquid at body temperature and therefore melt in the rectum or vaginal cavity and release the active compound.

Solid dosage forms for oral administration include capsules, tablets, pills, powders, and granules. In such solid dosage forms, the active compound is mixed with at least one inert, pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also comprise buffering agents.

Solid compositions of a similar type may also be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes. Solid compositions of a similar type may also be employed as fillers in soft and hard-filled gelatin capsules using such excipients as lactose or milk sugar as well as high molecular weight polethylene glycols and the like.

The active compounds can also be in micro-encapsulated form with one or more excipients as noted above. The solid dosage forms of tablets, dragees, capsules, pills, and granules can be prepared with coatings and shells such as enteric coatings, release controlling coatings and other coatings well known in the pharmaceutical formulating art. In such solid dosage forms the active compound may be admixed with at least one inert diluent such as sucrose, lactose or starch. Such dosage forms may also comprise, as is normal practice, additional substances other than inert diluents, e.g., tableting lubricants and other tableting aids such a magnesium stearate and microcrystalline cellulose. In the case of capsules, tablets and pills, the dosage forms may also comprise buffering agents. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes.

Dosage forms for topical or transdermal administration of a compound of this invention include ointments, pastes, creams, lotions, gels, powders, solutions, sprays, inhalants or patches. The active component is admixed under sterile conditions with a pharmaceutically acceptable carrier and any needed preservatives or buffers as may be required. Ophthalmic formulation, ear drops, and eye drops are also contemplated as being within the scope of this invention. Additionally, the present invention contemplates the use of transdermal patches, which have the added advantage of providing controlled delivery of a compound to the body. Such dosage forms can be made by dissolving or dispensing the compound in the proper medium. Absorption enhancers can also be used to increase the flux of the compound across the skin. The rate can be controlled by either providing a rate controlling membrane or by dispersing the compound in a polymer matrix or gel.

In certain embodiments, the present invention provides a pharmaceutical composition comprising X4P-001 or a pharmaceutically acceptable salt thereof, one or more diluents, a disintegrant, a lubricant, a flow aid, and a wetting agent. In some embodiments, the present invention provides a composition comprising 10-1200 mg X4P-001 or a pharmaceutically acceptable salt thereof, microcrystalline cellulose, dibasic calcium phosphate dihydrate, croscarmellose sodium, sodium stearyl fumarate, colloidal silicon dioxide, and sodium lauryl sulfate. In some embodiments, the present invention provides a unit dosage form wherein said unit dosage form comprises a composition comprising 10-200 mg X4P-001, or a pharmaceutically acceptable salt thereof, microcrystalline cellulose, dibasic calcium phosphate dihydrate, croscarmellose sodium, sodium stearyl fumarate, colloidal silicon dioxide, and sodium lauryl sulfate. In certain embodiments, the present invention provides a unit dosage form comprising a composition comprising X4P-001 or a pharmaceutically acceptable salt thereof, present in an amount of about 10 mg, about 20 mg, about 25 mg, about 50 mg, about 75 mg, about 100 mg, about 125 mg, about 150 mg, about 200 mg, about 250 mg, about 300 mg, about 400 mg, about 450 mg, about 500 mg, about 600 mg, about 650 mg, about 700 mg, about 750 mg, about 800 mg, about 850 mg, about 900 mg, about 950 mg, about 1000 mg, about 1100 mg, about 1200 mg, about 1300 mg, about 1400 mg, about 1500 mg, or about 1600 mg. In some embodiments, a provided composition (or unit dosage form) is administered to the patient once per day, twice per day, three times per day, or four times per day. In some embodiments, a provided composition (or unit dosage form) is administered to the patient once per day or twice per day. In some embodiments, the unit dosage form comprises a capsule containing about 25 mg, about 50 mg, about 75 mg, about 100 mg, about 150 mg, or about 200 mg of X4P-001 or a pharmaceutically acceptable salt thereof.

In some embodiments, the present invention provides a unit dosage form comprising a pharmaceutical composition comprising:
(a) X4P-001, or a pharmaceutically acceptable salt thereof as about 30-40% by weight of the composition;
(b) microcrystalline cellulose as about 20-25% by weight of the composition;
(c) dibasic calcium phosphate dihydrate as about 30-35% by weight of the composition;
(d) croscarmellose sodium as about 5-10% by weight of the composition;
(e) sodium stearyl fumarate as about 0.5-2% by weight of the composition;
(f) colloidal silicon dioxide as about 0.1-1.0% by weight of the composition; and
(g) sodium lauryl sulfate as about 0.1-1.0% by weight of the composition.

In some embodiments, the present invention provides a unit dosage form comprising a composition comprising:
(a) X4P-001, or a pharmaceutically acceptable salt thereof as about 37% by weight of the composition;
(b) microcrystalline cellulose as about 23% by weight of the composition;
(c) dibasic calcium phosphate dihydrate as about 32% by weight of the composition;
(d) croscarmellose sodium as about 6% by weight of the composition;
(e) sodium stearyl fumarate as about 1% by weight of the composition;
(f) colloidal silicon dioxide as about 0.3% by weight of the composition; and
(g) sodium lauryl sulfate as about 0.5% by weight of the composition.

In some embodiments, the present invention provides a unit dosage form comprising a composition comprising:
(a) X4P-001, or a pharmaceutically acceptable salt thereof as about 55-65% by weight of the composition;
(b) microcrystalline cellulose as about 10-15% by weight of the composition;
(c) dibasic calcium phosphate dihydrate as about 15-20% by weight of the composition;
(d) croscarmellose sodium as about 5-10% by weight of the composition;
(e) sodium stearyl fumarate as about 0.5-2% by weight of the composition;
(f) colloidal silicon dioxide as about 0.1-1.0% by weight of the composition; and
(g) sodium lauryl sulfate as about 0.1-1.0% by weight of the composition.

Inasmuch as it may be desirable to administer a combination of active compounds, for example, for the purpose of treating a particular disease or condition, it is within the scope of the present invention that two or more pharmaceutical compositions, at least one of which contains a compound in accordance with the invention, may conveniently be combined in the form of a kit suitable for co-administration of the compositions. Thus the kit of the invention includes two or more separate pharmaceutical compositions, at least one of which contains a compound of the invention, and means for separately retaining said compositions, such as a container, divided bottle, or divided foil packet. An example of such a kit is the familiar blister pack used for the packaging of tablets, capsules and the like.

The kit of the invention is particularly suitable for administering different dosage forms, for example, oral and parenteral, for administering the separate compositions at different dosage intervals, or for titrating the separate compositions against one another. To assist compliance, the kit typically includes directions for administration and may be provided with a memory aid.

The examples below explain the invention in more detail. The following preparations and examples are given to enable those skilled in the art to more clearly understand and to practice the present invention. The present invention, however, is not limited in scope by the exemplified embodiments, which are intended as illustrations of single aspects of the invention only, and methods which are functionally equivalent are within the scope of the invention. Indeed, various modifications of the invention in addition to those described herein will become apparent to those skilled in the art from the foregoing description and accompanying drawings. Such modifications are intended to fall within the scope of the appended claims.

The contents of each document cited in the specification are herein incorporated by reference in their entireties.

### EXEMPLIFICATION

### Example 1: A Clinical Trial of Mavorixafor, an Oral CXCR4 Antagonist, in Combination with Ibrutinib in Patients with Waldenstrom's Macroglobulinemia (WM) Whose Tumors Express Mutations in MYD88 and CXCR4

### Objectives:

Primary

As a primary objective of the study, mavorixafor and ibrutinib are administered in order to establish a pharmacologically active dose of mavorixafor in combination with ibrutinib based on pooled safety, clinical response, pharmacokinetic (PK), and pharmacodynamic (PD) data.

Secondary

As secondary objectives of the study:
clinical response indicators, including changes in serum immunoglobulin M (IgM) and hemoglobin (Hgb), are assessed from baseline through Cycle 6
the major response rate (defined as complete response+very good partial response+partial response) is assessed at the end of Cycle 6
the safety profile of the combination of mavorixafor and ibrutinib is assessed.

Exploratory

As a further exploratory objective, the effect of treatment with mavorixafor in combination with ibrutinib on selected PD and disease-related biomarkers is evaluated.

### Endpoints:

Primary

The primary endpoints are:
Safety: dose-limiting toxicities (DLTs)
Clinical response: percent change from baseline in IgM and Hgb during Cycles 1, 2, and 3
Pharmacokinetics: PK parameters of mavorixafor and ibrutinib during Cycles 1, 2 and 3
Pharmacodynamics: changes in area under the concentration-time curve (AUC) of absolute neutrophil count (ANC) and maximal change in ANC count, both compared with baseline during Cycles 1, 2, and 3

Secondary

The secondary endpoints are:
Percent changes in serum IgM levels from baseline through Cycle 6
Changes in Hgb from baseline through Cycle 6
Major response rate from baseline through Cycle 6 according to the criteria adopted from the Sixth International Workshop on Waldenstrom's Macroglobulinemia
Safety, assessed through the reporting of adverse events (AEs), findings on physical examination and other diagnostic procedures, the results of laboratory determinations, and use of concomitant medications; safety findings are recorded continuously throughout the study

Exploratory
Assessment of clinical responses in Cycles 7 through 24 including changes in serum IgM levels and Hgb levels

### Study Design:

This is a Phase 1b, open-label, multicenter, single-arm study of mavorixafor in combination with ibrutinib in patients with WM whose tumors express the MYD88 mutation as well as a variety of mutations in CXCR4. The primary objective is to establish a pharmacologically active dose of mavorixafor in combination with ibrutinib based on pooled safety, PK, and PD data to select the recommended dose for a randomized registrations trial.

This is an intrapatient dose-escalation study. Three dose levels of mavorixafor are explored: 200 mg QD (dose level 1), 400 mg QD (dose level 2), and 600 mg QD (dose level 3). Ibrutinib is administered at its labeled dose for patients with WM, 420 mg orally QD. Each treatment cycle is 28 days. Each patient will initially receive mavorixafor at 200 mg in combination with ibrutinib 420 mg. If the dose is well tolerated and there are no DLTs observed at dose level 1 during the 28 day DLT observation period, the patient will receive 400 mg QD in combination with ibrutinib during Cycle 2 for 28 days. If the patient does not experience a DLT at the 400 mg dose level at the end of Cycle 2, the patient will receive mavorixafor at 600 mg QD in combination with ibrutinib during Cycle 3. If the patient tolerates the 600 mg dose for 28 days and no DLTs are observed, the patient will continue to receive study treatment with mavorixafor at the 600 mg dose in combination with ibrutinib.

If during Cycle 1 (mavorixafor 200 mg QD), the patient experiences a DLT, the patient is withdrawn from the study and no further study drugs are administered to the patient.

If a patient experiences a DLT during Cycle 2 or Cycle 3, study drugs are withheld and dose de-escalation of mavorixafor to the previously cleared dose is permitted once the AE resolves to Grade 1 or returns to the baseline level of severity.

Once the maximum tolerated dose for the patient has been identified, the patient may continue to receive study treatment until progression of disease, an unacceptable toxicity, death, or study withdrawal for any other reasons for 2 years to assess for safety and clinical response at the maximum tolerated dose.

Any patient who has their dose of mavorixafor lowered during the study after Cycle 1 because of an AE may have the dose re-escalated. In the event the toxicity is attributed to ibrutinib in any patient, the ibrutinib dose may be reduced to 280 mg QD or to 140 mg QD (See IMBRUVICA® Prescribing Information).

Dose-limiting toxicities (DLT) are based on National Cancer Institute (NCI) Common Terminology Criteria for Adverse Events (CTCAE) version 5 and will consist of any treatment-related Grade 3 or Grade 4 nonhematologic toxicities other than inadequately treated nausea, vomiting, diarrhea, or electrolyte abnormalities reversed within 72 hours; Grade 4 thrombocytopenia: Grade 3 thrombocytopenia with bleeding, or Grade 4 neutropenia lasting >7 days. If ≥33% of patients develop a DLT, at a certain dose level, dose escalation of the remaining patients in the study to that dose level will stop.

The recommended dose for a randomization trial is based on the review of the safety, PK, PD data, and an integrated exposure-response (ER) analysis. Pharmacodynamic endpoints will include assessments of AUC of the ANC count and maximal change in ANC count.

Patients will undergo a screening period of up to 28 days before the first dose of combination therapy to ensure eligibility. Somatic mutation status of MYD88 and CXCR4 must be documented prior to the study treatment. All patients must fulfill the requirements for active treatment of WM based on the consensus panel criteria from the Eighth International Workshop on Waldenstrom's Macroglobulinemia.

Pharmacodynamic response is assessed after Cycles 1, 2, and 3. Clinical response is assessed based on IgM levels and Hbg levels. Patients may remain on the study and receive study drugs for up to 2 years after their highest tolerated dose is identified, in order to determine clinical response unless the patient develops progressive neoplastic disease, an unacceptable toxicity, death, or study withdrawal for any other reasons. Patients will continue to be followed for up to 30 days after last dose of study drugs. Patients who discontinue due to an AE are followed until the resolution of the AE or 30 days, whichever is longer.

Safety assessments are conducted including SAE and AE collection, physical examinations, vital sign measurements, assessment of Eastern Cooperative Oncology Group (ECOG) performance status, electrocardiograms (ECGs), and safety laboratory assessments.

An End-of-Treatment (EOT) visit is conducted within 7 days after the last dose of study treatment for patients who complete the planned study treatment and for patients who terminated prematurely for any reason. An End-of-Study (EOS) visit is conducted 30 days (±7 days) after the last dose of study treatment for safety assessments.

### Investigational Product, Dosage and Mode of Administration:

Mavorixafor is supplied as 100 mg capsules for daily oral administration. Doses explored in the study will include 200 mg QD, 400 mg QD, and 600 mg QD.

Ibrutinib is supplied as 140 mg capsules for daily oral administration.

### Duration of Study Participation:

The study is comprised of a 28-day screening period, up to 3 treatment cycles of 28 days each at 200 mg, 400 mg, and 600 mg QD, and may continue study treatment for up to 2 years at the highest tolerable dose. Patients will have a 30-day follow-up period after completion of treatment for a maximal duration of participation of approximately 26 months.

Table of Abbreviations

| Abbreviation or | |
|---|---|
| Specialist Term | Explanation |
| AE | Adverse event |
| AKI | Acute kidney injury |
| ANC | Absolute neutrophil count |
| AUC | Area under the concentration-time curve |
| BTK | Bruton's tyrosine kinase |
| Cₘₐₓ | Maximum observed concentration |
| CNS | Central nervous system |
| CTCAE | Common Terminology Criteria for Adverse Events |
| CXCR4 | Chemokine (C-X-C motif) receptor 4 |
| CXCL12 | Chemokine (C-X-C motif) 12 |
| CYP3A4 | Cytochrome P450 3A4 |
| DLT | Dose-limiting toxicity |
| ECG | Electrocardiogram |
| ECOG | Eastern Cooperative Oncology Group |
| eCRF | Electronic case report form |
| EOS | End-of-study |
| ER | Exposure response |
| EU | European Union |
| FDA | Food and Drug Administration |
| GCP | Good Clinical Practice |
| HbA1c | Hemoglobin A1c |
| Hgb | Hemoglobin |
| HIV | Human immunodeficiency virus |
| HSCs | Hematopoietic stem cells |
| ICH | International Conference on Harmonization |
| IEC | Independent Ethics Committee |
| IgM | Immunoglobulin M |
| IL | interleukin |
| IRB | Institutional Review Board |
| MedDRA | Medical Dictionary for Regulatory Activities |
| MGUS | Monoclonal gammopathy of undetermined significance |
| MYD88 | Myeloid differentiation primary response 88 |
| NCI | National Cancer Institute |
| PD | Pharmacodynamic |
| P-gp | P-glycoprotein |
| PK | Pharmacokinetic |
| PT | Preferred term |
| QD | Once daily |
| QTc | Corrected QT interval |
| SAE | Serious adverse event |
| SOC | System organ class |
| SUSAR | Suspected, unexpected serious adverse reaction |
| TEAE | Treatment-emergent adverse event |
| TLT | Treatment-limiting toxicity |
| TNF | Tumor necrosis factor |
| ULN | Upper limit of normal |
| US | United States |
| VGPR | Very good partial response |
| WBC | White blood cell |
| WHIM | Warts, hypogammaglobulinemia, infections, and |
| | myelokathexis |
| WM | Waldenstrom's Macroglobulinemia |
| WOCBP | Women of child-bearing potential |
| WT | Wild type |

### Rationale for Study Design

An intrapatient dose escalation design is conducted for this study to establish a pharmacologically active dose of mavorixafor in combination with ibrutinib based on both the safety of the combination and pharmacodynamic response. This design has the benefit of maximizing the likelihood that each patient is treated with an effective and safe dose, therefore enhancing the potential for clinical benefit while facilitating enrollment of patients with this rare disease.

The pharmacodynamic response of neutrophil changes to mavorixafor is rapid, occurring within hours to days. This characteristic of mavorixafor, allows for intrapatient patient dose escalation and since the steady-state ANC response is achieved rapidly as doses are escalated, the 28-day treatment duration cycle is intended to ensure that steady-state changes in ANC counts are achieved at each dose level, thereby avoiding any potential carry-over effects of previous doses. In addition, any DLT events are attributed to the current dose regardless of duration, making it unlikely that an unsafe dose is selected based on the algorithm.

### Rationale for Study Dose

Dose selection for the planned Phase 1b study is based on available safety data and PD of CXCR4 inhibition. The proposed starting dose (200 mg) is selected based on experience in prior clinical trials demonstrating that the dose was (a) pharmacologically active and (b) well-tolerated. An intrapatient dose escalation scheme is used in this study: patients will have a dose escalation in 200-mg increments to a maximum dose of 600 mg QD with the objective of achieving maximum white blood cell (WBC) mobilization.

Mavorixafor (Stone 2007) and the related CXCR4 antagonist, plerixafor (Hendrix 2000) cause a dose-dependent elevation in the WBC count that peaks approximately 3 to 6 hours after drug administration. The maximum fold-increase in WBCs was 3-fold over baseline and was similar across WBC subsets of neutrophils, monocytes, and lymphocytes, including B cells. Patients with WHIM who had mutations in CXCR4 exhibited a greater than 40-fold increase in CD19 B cells in response to plerixafor (Dale 2011). Mobilization of the B-cell population from the bone marrow micro-environment may be an important mechanism that sensitizes tumor cells in WM patients to the cytotoxic effects of ibrutinib and WBC mobilization may be viewed as a biomarker of maximal CXCR4 inhibition. Both WBC mobilization and the magnitude of the response is maintained when mavorixafor is combined with a kinase inhibitor, axitinib, in renal cell carcinoma patients, with the 600 mg QD dose causing a peak 3-fold elevation compared with the 400-mg QD dose (mean fold increase approximately 2-fold). This supports an increment in the on-target biologic effect of 600 mg versus 400 mg. As CXCR4 mutations in WM are considered to be oncogenic drivers, administering the MTD is considered necessary to maximize potential efficacy. The doses selected in the current Phase 1b trial in WM patients are therefore intended to achieve maximum inhibition of CXCR4 at 600 mg QD without the need for further dose escalation.

Doses of mavorixafor up to 600 mg daily have been tested in other clinical trials. During a Phase 1/2 study in renal cell carcinoma, a 3-patient cohort received monotherapy with 600 mg mavorixafor with 2 DLTs reported. On review, one of these (hyperglycemia) occurred in a patient with undiagnosed diabetes mellitus and the other (acute kidney injury [AKI]) occurred in a patient with pre-existing renal dysfunction and other complicating factors. Six patients received the 600 mg mavorixafor dose together with axitinib. Two of these patients reported DLTs (Grade 2 cognitive impairment; Grade 3 fatigue and dyspnea), while a third patient reported an SAE of AKI that was not considered a DLT. In-depth review of these cases resulted in uncertainty as to which agent in the combination therapy may have been responsible for the DLTs, while the patient with AKI underwent rechallenge without recurrence of AKI. Patients with a high hemoglobin A1c (HbA1c) level or with renal dysfunction are excluded from this study for safety reasons.

### Patient Inclusion Criteria

Patients with a clinical diagnosis of WM must meet all the following criteria to be eligible for study participation:
1. Patients must be at least 18 years of age
2. Patients or their legal representative must be able to sign informed consent
3. Patients must have a clinicopathological diagnosis of WM and must meet the criteria for treatment using consensus panel criteria from the Second International Workshop on Waldenstrom's Macroglobulinemia
4. Patients' WM must have confirmed MYD88^{L265P} and CXCR4^{WHIM} mutations
5. Patients must have measurable disease, defined as the presence of serum IgM with a minimum IgM level of ≥2×the upper limit of normal (ULN)
6. Patients may be treatment naïve or have received up to 3 prior treatment regimens for WM
7. Patients must have an ECOG performance status of 0 or 1
8. Patients must meet the following organ and bone marrow requirements:
   a. Absolute neutrophil count ≥1,000/μL
   b. Platelet count ≥50,000/μL (platelet transfusion-independent)
   c. Hgb ≥8 gm/dL
   d. Aspartate aminotransferase and alanine aminotransferase ≤2.5×the ULN AND serum total bilirubin ≤1.5×the ULN, unless secondary to known Gilbert's Syndrome or hepatic infiltration by WM, in which case the total bilirubin must be ≤3×the ULN and direct bilirubin ≤1.5×the ULN
   e. Serum lipase ≤1.5×the ULN
   f. Serum creatinine ≤2×the ULN or a creatinine clearance of ≥30 ml/minute based on the Cockcroft-Gault equation
9. Women of child-bearing potential (WOCBP) must have a negative pregnancy test
10. WOCBP who are heterosexually active and male patients with female sexual partners of childbearing potential must agree to use an effective method of contraception (e.g., oral contraceptives, double-barrier methods such as a condom and a diaphragm, intrauterine device) during the study and for 4 weeks after the last dose of study medication, or to abstain from sexual intercourse for this time; a woman not of childbearing potential is one who has undergone a bilateral oophorectomy or who is postmenopausal, defined as the absence of menstrual periods for 12 consecutive months
11. Patients must be willing and capable of complying with the requirements of the study

### Patient Exclusion Criteria

Patients with any of the following are excluded from participation in the study:
1. Patients with hyperviscosity syndrome; patients who undergo plasmapheresis for hyperviscosity may be considered for enrollment once IgM level is under 4,000 mg/dl
2. Patients who have known hypersensitivity to mavorixafor or any of its components or to ibrutinib
3. Patients who have previously received a CXCR4 inhibitor or a BTK inhibitor
4. Patients who are pregnant or breastfeeding
5. Patients with an infection requiring intravenous antibiotics or hospitalization at the scheduled time of the first administration of protocol therapy
6. Patients with HbA1c≥6.5%
7. Patients with central nervous system (CNS) lymphoma; patients with suspected CNS lymphoma should undergo appropriate diagnostic studies (magnetic resonance imaging, lumbar puncture) before enrollment to determine if CNS lymphoma is present
8. Patients with ongoing acute clinical AEs of NCI CTCAE Grade >1 resulting from prior cancer therapies or patients receive prior chemotherapy within 2 weeks of initial dosing or prior autologous hematopoietic stem cell transplantation (auto-HSCT) within 6 weeks of initial dosing
9. Patients with a history of, or positive serologies for, HIV, hepatitis B or hepatitis C infection (patients with HBsAb positivity due to a HBV vaccination are eligible)
10. Patients who have had within the past 6 months, the occurrence or persistence of one or more of the following medical conditions that could not be controlled with usual medical care (e.g., required emergency care or hospitalization): hypertension, diabetes, unstable angina, seizure disorder, or myocardial infarction
11. Patients with clinically significant cardiac disease, including congestive heart failure consistent with New York Heart Association Class 3 or 4; uncontrolled hypertension, clinically significant angina, clinically significant arrythmias including a history of atrial fibrillation, corrected QT interval using Fridericia formula of >470 msec or a history of prolonged QT syndrome
12. Patients who have had within the past 6 months the occurrence of one or more of the following events: cerebrovascular accident, deep vein thrombosis, pulmonary embolism, hemorrhage (NCI CTCAE Grade 3 or Grade 4), or chronic liver disease (meeting criteria for Child-Pugh Class B or C)
13. Patients with prior organ transplantation (Prior auto-HSCT is eligible)
14. Patients who have a known bleeding disorder or require an anticoagulant at the time of study treatment
15. Patients with active autoimmune disease requiring systemic steroid administration
16. Patients with active second malignancies (except: malignancies that were treated curatively and have not recurred within 2 years prior to study treatment; completely resected basal cell and squamous cell skin cancers; any non-hematological malignancy considered to be indolent and that has never required therapy; and completely resected carcinoma in situ of any type
17. Patients who have received an investigational agent within 5 half-lives of the agent; if the half-life of the agent is unknown, patients must wait 4 weeks
18. Patients who require strong or moderate inhibitors or inducers of CYP3A4 and potent P-gp inhibitors
19. Patients who require medications which are classified as sensitive CYP2D6 substrates
20. Patient who have received in the 2 weeks preceding the first dose of protocol treatment, any of the following agents:
Granulocyte colony-stimulating factor or granulocyte-macrophage colony-stimulating factor
Systemic corticosteroids in a dose of >10 mg equivalent of prednisone daily; topical, ophthalmic, intranasal, and inhalational corticosteroids are permitted
Any other immunomodulating agents, including but not limited to interferon alpha, interleukin (IL)-2, mycophenolate, antibodies to tumor necrosis factor (TNF)-α, soluble TNF receptors, Janus kinase inhibitors, or IL-23 antagonists
21. Patients with any other medical, personal, social, or psychiatric condition that, in the opinion of the Investigator, may potentially compromise the safety or compliance of the patient or precludes the patient's participation in the study.

### Description of Study Drugs

Mavorixafor is a second-generation, small-molecule, non-competitive, allosteric antagonist of CXCR4 that acts by binding to extracellular domains of the receptor, resulting in specific and reversible inhibition of receptor signaling in response to its ligand CXCL12.

IMBRUVICA® (ibrutinib) is new class of orally administered selective and irreversible small-molecule inhibitor of BTK. It forms a covalent bond with a cysteine 481 in the catalytic kinase domain. This leads to inhibition of BTK enzymatic activity.

### Dosing

This is an intrapatient dose-escalation study. Three dose levels of mavorixafor is administered in doses of 200 mg, 400 mg, and 600 mg orally QD.

Ibrutinib is administered at its labeled dose in WM of 420 mg orally QD.

### Study Drug Administration

Patients will receive mavorixafor capsules (100-mg dose strength) and ibrutinib (140-mg dose strength) orally QD.

Patients are instructed about both the dosing schedule and requirements relating to food or drink near the time of dosing.

The first dose of study drug is taken at the study center under the observation of study center personnel.

### Dosing Schedule

It is expected that the daily dose of mavorixafor and ibrutinib are taken as follows:

The daily dose should be taken in the morning, at the same time each day (±2 hours) with water.

The interval between successive doses should not be <21 hours nor >27 hours.

If the interval between successive doses is <16 hours before the next scheduled dose time, the dose should be omitted, and the usual schedule resumed the next day.

At office visits for PK/PD sampling, dosing must be held in the morning, until after the predose or time 0 blood samples have been obtained.

Capsules should not be cut, crushed, or chewed.

Extra doses of mavorixafor or ibrutinib should not be taken to make up for missed doses.

### Food Restrictions

Absorption of mavorixafor is reduced when taken with food, whereas ibrutinib absorption is increased. Patients are instructed to take both mavorixafor and ibrutinib together as follows:

No food or drink (except water) for 1 hour predose, and

No food or drink (except water) for 2 hours postdose.

Patients for whom the scheduling requirements and eating restrictions represent significant difficulties should be discussed with the Medical Monitor to develop the most effective regimen possible.

### Dose Modifications

If a patient receiving study treatment (mavorixafor) successfully completes the first 3 cycles of treatment, and subsequently has a TLT event, study treatment may, with agreement of the patient, Investigator, and Medical Monitor, be managed.

### REFERENCES

1. Buske C, Leblond V, Dimopoulos M, et al. Waldenstrom's macroglobulinaemia: ESMO Clinical Practice Guidelines for diagnosis, treatment and follow-up. Ann Oncol. 2013; 24(6):vi155-9.
2. Cao Y, Hunter Z, Liu X, et al. The WHIM-like CXCR4(S338X) somatic mutation activates AKT and ERK, and promotes resistance to ibrutinib and other agents used in the treatment of Waldenstrom's Macroglobulinemia. Leukemia. 2014; 29(1):169-76.
3. Cao Y, Hunter Z, Liu X, et al. CXCR4 WHIM-like frameshift and nonsense mutations promote ibrutinib resistance but do not supplant MYD88(L265P)-directed survival signaling in Waldenstrom macroglobulinaemia cells. Br J Haematol. 2015; 168(5):701-7.
4. Castillo J, Olszewski A, Kanan S, et al. Overall survival and competing risks of death in patients with Waldenstrom macroglobulinaemia: an analysis of the Surveillance, Epidemiology and End Results database. Br J Haematol. 2015; 169(1):81-9.
5. Chatterjee S, Azad B, Nimmagadda S. The intricate role of CXCR4 in cancer. Adv Cancer Res. 2014; 124:31-82.
6. Christiansen C F, Johansen M B, Langeberg W J, et al. Incidence of acute kidney injury in cancer patients: a Danish population-based cohort study. Eur J Intern Med. 2011; 4:399-406.
7. Dale D, Bolyard A, Kelley M, et al. The CXCR4 antagonist plerixafor is a potential therapy for myelokathexis, WHIM syndrome. Blood. 2011; 118(18):4963-66. doi:10.11.1182/blood-2011-06-360586
8. de Duve C, de Barsy T, Poole B, et al. Lysosomotropic agents. Biochem Pharmacol. 1974; 23:2495-2534.
9. De Falco V, Guarino V, Avilla E, et al. Biological role and potential therapeutic targeting of the chemokine receptor CXCR4 in undifferentiated thyroid cancer. Cancer Res 2007; 67:11821-9.
10. Dimopoulos M, Panayiotidis P, Moulopoulos L, et al. Waldenstrom's macroglobulinemia: clinical features, complications, and management. J Clin Oncol. 2000; 18(1):214-26.
11. Dimopoulos M, Anagnostopoulos A, Kyrtsonis M, et al. Primary treatment of Waldenstrom macroglobulinemia with dexamethasone, rituximab, and cyclophosphamide. J Clin Oncol. 2007; 25(22):3344-9.
12. Dimopoulos M, Kastritis E, Owen R, et al. Treatment recommendations for patients with Waldenstrom macroglobulinemia (WM) and related disorders: IWWM-7 consensus. Blood. 2014; 124(9):1404-11.
13. Dimopoulos M, Trotman J, Tedeschi A, et al. Ibrutinib for patients with rituximab-refractory Waldenstrom's macroglobulinaemia (INNOVATE): an open-label substudy of an international, multicentre, Phase 3 trial. Lancet Oncol. 2017; 18(2):241-250.
14. Dimopoulos M, Tedeschi A, Trotman J, et al. Phase 3 Trial of Ibrutinib plus Rituximab in Waldenstrom's Macroglobulinemia. N Engl J Med. 2018; 378(25):2399-2410.
15. FDA Drug development and drug interactions: table of substrates, inhibitors and inducers. Updated Nov. 14, 2017. Available at fda.gov/drugs/drug-interactions-labeling/drug-development-and-drug-interactions-table-substrates-inhibitors-and-inducers. Accessed Jun. 22, 2019.
16. Fonseca R, Hayman S. Waldenstrom macroglobulinaemia. Br J Haematol. 2007; 138(6):700-20.
17. Goldman J, Mendenhall M, Rettinger S. Hyperglycemia associated with targeted oncologic treatment: mechanisms and management. The Oncologist. 2016; 21:1326-36.
18. Groves F, Travis L, Devesa S, et al. Waldenstrom's macroglobulinemia: incidence patterns in the United States, 1988-1994. Cancer. 1998; 82(6):1078-81.
19. Hassan S, Buchanan M, Jahan K, et al. CXCR4 peptide antagonist inhibits primary breast tumor growth, metastasis and enhances the efficacy of anti-VEGF treatment or docetaxel in a transgenic mouse model. Int J Cancer 2010; 129:225-32.
20. Hendrix C, Flexner C, MacFarland R, et al. Pharmacokinetics and safety of AMD-3100, a novel antagonist of the CXCR-4 cytokine receptor, in human volunteers. Antimicrob Agent and Chemother. 2000; 44(6):1167-73.
21. Hruban Z. Pulmonary changes induced by amphophilic drugs. Environ Health Perspect. 1976; 16:111-18
22. Huang E H, Singh B, Cristofanilli M, et al. A CXCR4 antagonist CTCE-9908 inhibits primary tumor growth and metastasis of breast cancer. J Surg Res 2009; 155:231-6.
23. Hunter Z, Xu L, Yang G, et al. The genomic landscape of Waldenstrom macroglobulinemia is characterized by highly recurring MYD88 and WHIM-like CXCR4 mutations, and small somatic deletions associated with B-cell lymphomagenesis. Blood. 2014; 123(11):1637-46.
24. IMBRUVICA® [Highlights of Prescribing Information]. Horsham, PA: Janssen Biotech, Inc; 2019. imbruvica.com/docs/librariesprovider7/default-document-library/prescribing-information.pdf
25. Ji Y, Jin J, Hyman D, et al. Challenges and opportunities in dose finding in oncology and immuno-oncology. Clin Transl Sci. 2018; 11:345-51.
26. Jiang H, Passarelli P, Munro G, et al. High-resolution sub-cellular imaging by correlative NanoSIMS and electron microscopy of amiodorone internalisation by lung macrophages as evidence for drug-induced phospholipidosis. Chem Commun. 2017; 53:1506-1509.
27. Kim S Y, Lee C H, Midura B V, et al. Inhibition of the CXCR4/CXCL12 chemokine pathway reduces the development of murine pulmonary metastases. Clin Exp Metastasis 2008; 25:201-11.
28. Kodovanti U, Mehendale H. Cationic amphiphilic drugs and phospholipid storage disorder. Pharmcol Review. 1990; 42(4):327-54.
29. Kyle R A, Therneau T M, Rajkumar S V, et at. Long-term follow-up of 241 patients with monoclonal gammopathy of undetermined significance: the original Mayo Clinic series 25 years later. Mayo Clin Proc. 2004; 79:859-65.
30. Kyle R A, Rajkumar S V. Monoclonal gammopathy of undetermined significance. Clin Lymphoma Myeloma. 2005; 6:102-14.
31. Lahoti A, Humphreys B. Chapter 3 AKI associated with malignancies. Amer Soc Nephrol. 2016; 3:1-8.
32. Lei N, Rubin E, Mehrotra N, et al. Rendering the 3+3 design to rest: more efficient approaches to oncology dose-finding trials in the era of targeted therapy. Clin Cancer Resch. 2016; 22(11):2623-9.
33. Liu Q, Li Z, Gao J L, et al. CXCR4 antagonist AMD3100 redistributes leukocytes from primary immune organs to secondary immune organs, lung, and blood in mice. Eur J Immunol. 2015:45(6):1855-67.
34. Liu Q, Pan C, Lopez L, et al. WHIM syndrome caused by Waldenstrom's macroglobulinemia-associated mutation CXCR4 (L329fs). J Clin Immunol. 2016; 36(4):397-405.
35. Majumdar S, Murphy P M. Adaptive immunodeficiency in WHIM syndrome. Int J Mol Sci. 2018; 20(1):3. Published 2018. doi:103390/ijms20010003
36. Maker A V, Yang, J C, Sherry R M, et al. Intrapatient dose escalation of anti-CTLA-4 antibody in patients with metastatic melanoma. J Immunother. 2006; 29(4):455-463.
37. Mazzucchelli M, Frustaci A, Deodato M, et al. Waldenstrom's macroglobulinemia: an update. Mediterr J Hematol Infect Dis. 2018; 10(1) e2018004. doi:10.4084/MJHID.2018.004
38. Moyle G, DeJesus E, Boffito M, et al. Proof of activity with AMD11070, an orally bioavailable inhibitor of CXCR4 tropic HIV type 1. Clin Infect Dis. 2009; 48(6):798-805.
39. Muller A, Homey B, Soto H, et al. Involvement of chemokine receptors in breast cancer metastasis. Nature 2001; 410:50-6.
40. Murugan R, Karajala-Subramanyam V, Lee M, et al. Acute Kidney injury in non-severe pneumonia is associated with an increased immune response and lower survival. Kidney Int. 2010; 77(6):527-35. doi:10.1038/ki.2009.502
41. Nagase H, Miyamasu M, Yamaguchi M, et al. Expression of CXCR4 in eosinophils: functional analyses and cytokine-mediated regulation. J Immunol. 2000; 164(11):5935-43.
42. Nowakowski G S, Witzig T E, Dinghi D, et al. Circulating plasma cells detected by flow cytometry as a predictor of survival in 302 patients with newly diagnosed multiple myeloma. Blood. 2005; 106:2276-79.
43. Nyunt M, Becker S, MacFarland R, et al. Pharmacokinetic Effect of AMDO70, an Oral CXCR4 Antagonist, on CYP3A4 and CYP2D6 Substrates Midazolam and Dextromethorphan in Healthy Volunteers. J Acquir Immune Defic Syndr. 2008; 47(5):559-565.
44. Olszewski A, Treon S, Castillo J. Evolution of Management and Outcomes in Waldenstrom Macroglobulinemia: A Population-Based Analysis. Oncologist. 2016; 21(11):1377-1386.
45. Owen R, Treon S, Al-Katib A, et al. Clinicopathological definition of Waldenstrom's macroglobulinemia: consensus panel recommendations from the Second International Workshop on Waldenstrom's Macroglobulinemia. Semin Oncol. 2003; 30(2):110-5.
46. Owen R, Pratt G, Auer R, et al. Guidelines on the diagnosis and management of Waldenstrom macroglobulinaemia. Br J Haematol. 2014; 165(3):316-33.
47. Porvasnik S, Sakamoto N, Kusmartsev S, et al. Effects of CXCR4 antagonist CTCE-9908 on prostate tumor growth. Prostate 2009; 69:1460-9.
48. Pophali P, Bartley A, Kapoor P, et al. Prevalence and survival of smoldering Waldenstrom macroglobulinaemia in the United States. Br J Haematol. 2018. 1011-70.
49. Poulain S, Roumier C, Venet-Caillault A, et al. Genomic Landscape of CXCR4 Mutations in Waldenstrom Macroglobulinemia. Clin Cancer Res. 2016; 22(6):1480-8.
50. Roccaro A, Sacco A, Himenez C, et al. C1013G/CXCR4 acts as a driver mutation of tumor progression and modulator of drug resistance in lymphoplasmacytic lymphoma. Blood. 2014; 123(26):4120-31.
51. Rubin J B, Kung A L, Klein R S, et al. A small-molecule antagonist of CXCR4 inhibits intracranial growth of primary brain tumors. Proc Nat Acad Sci USA 2003; 100:13513-8.
52. Sacco A, Fenotti A, Affo, L, et al. The importance of the genomic landscape in Waldenstrom's macroglobulinemia of targeted therapeutical intervention. Oncotarget. 2017; 8:35435-44.
53. Sarma J S M, Pei H, Venkataraman K. Role of oxidative stress in amiodorone-induced toxicity. J Cardiovasc Pharmacol Therapeut. 1997; 2(1):53-60.
54. Schimanski C C, Bahre R, Gockel I, et al. Dissemination of hepatocellular carcinoma is mediated via chemokine receptor CXCR4. Br J Cancer 2006; 95:210-7.
55. Smith M C, Luker K E, Garbow J R, et al. CXCR4 regulates growth of both primary and metastatic breast cancer. Cancer Res 2004; 64:8604-12.
56. Stone N, Dunaway S, Flexner C, et al. Multiple-dose escalation study of the safety, pharmacokinetics and biologic activity of oral AMDO70, a selective CXCR4 receptor inhibitor, in human subjects. Antimicrob Agent and Chemother. 2007; 51(7):2351-58/-73.
57. Treon S, XIII. Waldenstrom's macroglobulinaemia: an indolent B-cell lymphoma with distinct molecular and clinical features. Hematol Oncol. 2013; 31 (1):76-80.
58. Treon S, Cao Y, Xu L, et al. Somatic mutations in MYD88 and CXCR4 are determinants of clinical presentation and overall survival in Waldenstrom macroglobulinemia. Blood. 2014; 123(18):2791-6.
59. Treon S, Tripsas C, Meid K, et al. Ibrutinib in previously treated Waldenstrom's macroglobulinemia. N Engl J Med. 2015; 372(15):1430-40. [1]
60. Treon S, How I treat Waldenstrom macroglobulinemia. Blood. 2015; 126(6):721-32. [2]
61. Treon S, Gustine J, Meid K, et al. Ibrutinib monotherapy in symptomatic, treatment-naive patients with Waldenstrom macroglobulinemia. J Clin Oncol. 2018; 36(27):2755-2761. [1]
62. Treon S, Meid K, Gustine J, et al. Ibrutinib shows prolonged progression-free survival in symptomatic, previously treated patients with MYD88 mutated Waldenstrom's macroglobulinemia: long-term follow-up of pivotal trial (NCT01614821). Abstr. PS1185. 23rd Conf Euro Hem Assoc. Jun. 16, 2018. Stockholm, Sweden. [2]
63. Varettoni M, Zibellini S, Defrancesco I, et al. Pattern of somatic mutations in patients with Waldenstrom macroglobulinemia or IgM monoclonal gammopathy of undetermined significance. Haematologica. 2017; 102(12):2077-2085.
64. Xu L, Hunter Z, Tsakmaklis N, et al. Clonal architecture of CXCR4 WHIM-like mutations in Waldenstrom Macroglobulinaemia. Br J Haematol. 2016; 172(5):735-44.
65. Zeelenberg I S, Ruuls-Van Stalle L E, Roos E. The chemokine receptor CXCR4 is required for outgrowth of colon carcinoma micrometastases. Cancer Res 2003; 63:3833-9.

We claim:

1. A method of treating Waldenstrom's macroglobulinemia (WM) in a patient in need thereof, comprising administering to the patient an effective amount of X4P-001, or a pharmaceutically acceptable salt thereof; in combination with an effective amount of a BTK inhibitor, or a pharmaceutically acceptable salt thereof; and wherein the WM bears one or more somatic mutations in the CXCR4 gene.

2. The method of claim 1, wherein the mutations comprise a gain of function mutation relative to wild type CXCR4.

3. The method of claim 1, wherein the mutations comprise a WHIM-like mutation that results in a gain of function in CXCR4 relative to wild type CXCR4.

4. The method of claim 1, wherein the mutations comprise a CXCR4(S338X) somatic mutation.

5. The method of any one of claims 1-4, wherein the WM further comprises a somatic MYD88 mutation and optionally a somatic deletion associated with B-cell lymphomagenesis.

6. The method of claim 5, wherein the MYD88 mutation is MYD88^{L265P}.

7. The method of any one of claims 1-6, wherein the BTK inhibitor is ibrutinib, acalabrutinib, or zanubrutinib.

8. The method of claim 7, wherein the BTK inhibitor is ibrutinib.

9. The method of claim 7, wherein the patient has previously received at least one course of treatment with a BTK inhibitor, or a pharmaceutically acceptable salt thereof, before treatment with X4P-001, or a pharmaceutically acceptable salt thereof.

10. The method of any one of claims 1-9, wherein the patient's WM is resistant to treatment with a BTK inhibitor.

11. The method of claim 1-9, wherein the patient has previously received at least one course of treatment with ibrutinib before treatment with X4P-001 or a pharmaceutically acceptable salt thereof.

12. The method of claim 11, wherein the patient's WM has shown disease progression.

13. The method of any one of claims 1-12, wherein X4P-001 or a pharmaceutically acceptable salt thereof is administered to the patient in a dose of about 100 mg to about 1000 mg per day.

14. The method of any one of claims 1-12, wherein X4P-001 or a pharmaceutically acceptable salt thereof is administered to the patient in a dose of about 200 mg to about 600 mg per day.

15. The method of any one of claims 1-12, wherein X4P-001 or a pharmaceutically acceptable salt thereof is administered to the patient in a dose of about 200 mg, about 400 mg, or about 600 mg per day.

16. The method of any one of claims 1-15, wherein X4P-001 or a pharmaceutically acceptable salt thereof is administered to the patient in a single daily dose (QD).

17. The method of any one of claims 1-16, wherein ibrutinib or a pharmaceutically acceptable salt thereof is administered to the patient in a dose of about 70 mg to about 840 per day.

18. The method of any one of claims 1-16, wherein ibrutinib or a pharmaceutically acceptable salt thereof is administered to the patient in a dose of about 140 mg to about 420 mg per day.

19. The method of any one of claims 1-16, wherein ibrutinib or a pharmaceutically acceptable salt thereof is administered to the patient in a dose of about 140 mg, about 280, or about 420 mg per day.

20. The method of any one of claims 1-16, wherein X4P-001 is administered to the patient in a dose of about 200 mg, about 400 mg, or about 600 mg per day, and ibrutinib is administered to the patient in a dose of about 140 mg, about 280 mg, or about 420 mg per day.

21. The method of any one of claims 1-19, wherein the method provides about a 75-95% percent reduction in IgM from baseline.

22. The method of any one of claims 1-19, wherein the method reduces IgM to within 2 times the normal range for a non-diseased adult human (non-WM patient).

23. The method of any one of claims 1-22, wherein the X4P-001, or a pharmaceutically acceptable salt thereof, and the BTK inhibitor, or a pharmaceutically acceptable salt thereof, act synergistically.

24. The method of claim 23, wherein the dose of the BTK inhibitor, or a pharmaceutically acceptable salt thereof, required for effective treatment is decreased by at least 20% relative to the effective dose of the BTK inhibitor, or a pharmaceutically acceptable salt thereof, as a monotherapy.

25. The method of any one of claims 1-24, further comprising the step of obtaining a biological sample from the patient and measuring the amount of a disease-related biomarker.

26. The method of claim 25, wherein the biological sample is a blood sample.

27. The method of claim 25, wherein the disease-related biomarker is selected from circulating CD8+ T cells or the ratio of CD8+ T cells:Treg cells.

28. The method of claim 25, wherein the disease-related biomarker is IgM and/or Hgb.

29. A method of determining whether a patient's WM will respond to treatment, comprising:
(a) testing a biological sample taken from the patient for a CXCR4 mutation and optionally a MYD88 mutation;
(b) if the patient's WM bears at least one CXCR4 mutation, selecting the patient for treatment with a CXCR4 inhibitor.

30. The method of claim 29, further comprising the step of treating the patient with a combination of an effective amount of X4P-001, or a pharmaceutically acceptable salt thereof; and an effective amount of a BTK inhibitor, or a pharmaceutically acceptable salt thereof, if the patient's WM bears at least one CXCR4 mutation.
